SEQUENCE LISTING (85 sequences)

SEQ ID NO: 1 - protein, 5 aa, synthetic construct
  REGION 1..5: note = hu CD40 CDR-H1
GYYIH

SEQ ID NO: 2 - protein, 17 aa, synthetic construct
  REGION 1..17: note = hu CD40 CDR-H2
RVIPNAGGTSYNQKFKG

SEQ ID NO: 3 - protein, 5 aa, synthetic construct
  REGION 1..5: note = hu CD40 CDR-H3
EGIYW

SEQ ID NO: 4 - protein, 16 aa, synthetic construct
  REGION 1..16: note = hu CD40 CDR-L1
RSSQSLVHSNGNTFLH

SEQ ID NO: 5 - protein, 7 aa, synthetic construct
  REGION 1..7: note = hu CD40 CDR-L2
TVSNRFS

SEQ ID NO: 6 - protein, 9 aa, synthetic construct
  REGION 1..9: note = hu CD40 CDR-L3
SQTTHVPWT

SEQ ID NO: 7 - protein, 114 aa, synthetic construct [sequence omitted]

SEQ ID NO: 8 - protein, 112 aa, synthetic construct [sequence omitted]

SEQ ID NO: 9 - protein, 5 aa, synthetic construct
  REGION 1..5: note = FAP (212) CDR-H1
DYNMD

SEQ ID NO: 10 - protein, 17 aa, synthetic construct
  REGION 1..17: note = FAP (212) CDR-H2
DIYPNTGGTIYNQKFKG

SEQ ID NO: 11 - protein, 10 aa, synthetic construct
  REGION 1..10: note = FAP (212) CDR-H3
FRGIHYAMDY

SEQ ID NO: 12 - protein, 15 aa, synthetic construct
  REGION 1..15: note = FAP (212) CDR-L1
RASESVDNYGLSFIN

SEQ ID NO: 13 - protein, 7 aa, synthetic construct
  REGION 1..7: note = FAP (212) CDR-L2
GTSNRGS

SEQ ID NO: 14 - protein, 9 aa, synthetic construct
  REGION 1..9: note = FAP (212) CDR-L3
QQSNEVPYT

SEQ ID NO: 15 - protein, 119 aa, synthetic construct
  REGION 1..119: note = humanized FAP (212) VH
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSS

SEQ ID NO: 16 - protein, 111 aa, synthetic construct
  REGION 1..111: note = humanized FAP (212) VL
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIK

SEQ ID NO: 17 - protein, 5 aa, synthetic construct
  REGION 1..5: note = FAP(28H1) CDR-H1
SHAMS

SEQ ID NO: 18 - protein, 16 aa, synthetic construct
  REGION 1..16: note = FAP(28H1) CDR-H2
AIWASGEQYYADSVKG

SEQ ID NO: 19 - protein, 8 aa, synthetic construct
  REGION 1..8: note = FAP(28H1) CDR-H3
GWLGNFDY

SEQ ID NO: 20 - protein, 12 aa, synthetic construct
  REGION 1..12: note = FAP(28H1) CDR-L1
RASQSVSRSYLA

SEQ ID NO: 21 - protein, 7 aa, synthetic construct
  REGION 1..7: note = FAP(28H1) CDR-L2
GASTRAT

SEQ ID NO: 22 - protein, 9 aa, synthetic construct
  REGION 1..9: note = FAP(28H1) CDR-L3
QQGQVIPPT

SEQ ID NO: 23 - protein, 116 aa, synthetic construct
  REGION 1..116: note = FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 24 - protein, 108 aa, synthetic construct
  REGION 1..108: note = FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 25 - protein, 5 aa, synthetic construct
  REGION 1..5: note = FAP(4B9) CDR-H1
SYAMS

SEQ ID NO: 26 - protein, 17 aa, synthetic construct
  REGION 1..17: note = FAP(4B9) CDR-H2
AIIGSGASTYYADSVKG

SEQ ID NO: 27 - protein, 8 aa, synthetic construct
  REGION 1..8: note = FAP(4B9) CDR-H3
GWFGGFNY

SEQ ID NO: 28 - protein, 12 aa, synthetic construct
  REGION 1..12: note = FAP(4B9) CDR-L1
RASQSVSRSYLA

SEQ ID NO: 29 - protein, 7 aa, synthetic construct
  REGION 1..7: note = FAP(4B9) CDR-L2
VGSRRAT

SEQ ID NO: 30 - protein, 9 aa, synthetic construct
  REGION 1..9: note = FAP(4B9) CDR-L3
QQGIMLPPT

SEQ ID NO: 31 - protein, 117 aa, synthetic construct
  REGION 1..117: note = FAP(4B9) VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 32 - protein, 108 aa, synthetic construct
  REGION 1..108: note = FAP(4B9) VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 33 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 34 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 35 - protein, 748 aa, synthetic construct
  REGION 1..748: note = hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 36 - protein, 761 aa, Mus sp.
  source 1..761: note = murine
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 37 - protein, 749 aa, synthetic construct
  REGION 1..749: note = Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 38 - protein, 748 aa, synthetic construct
  REGION 1..748: note = Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 39 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 40 - protein, 5 aa, synthetic construct
  REGION 1..5: note = Peptide linker G4S
GGGGS

SEQ ID NO: 41 - protein, 10 aa, synthetic construct
  REGION 1..10: note = Peptide linker (G4S)2
GGGGSGGGGS

SEQ ID NO: 42 - protein, 10 aa, synthetic construct
  REGION 1..10: note = Peptide linker (SG4)2
SGGGGSGGGG

SEQ ID NO: 43 - protein, 14 aa, synthetic construct
  REGION 1..14: note = Peptide linker G4(SG4)2
GGGGSGGGGSGGGG

SEQ ID NO: 44 - protein, 10 aa, synthetic construct
  REGION 1..10: note = Peptide linker
GSPGSSSSGS

SEQ ID NO: 45 - protein, 15 aa, synthetic construct
  REGION 1..15: note = Peptide linker (G4S)3
GGGGSGGGGSGGGGS

SEQ ID NO: 46 - protein, 20 aa, synthetic construct
  REGION 1..20: note = Peptide linker (G4S)4
GGGGSGGGGSGGGGSGGGGS

SEQ ID NO: 47 - protein, 8 aa, synthetic construct
  REGION 1..8: note = Peptide linker
GSGSGSGS

SEQ ID NO: 48 - protein, 8 aa, synthetic construct
  REGION 1..8: note = Peptide linker
GSGSGNGS

SEQ ID NO: 49 - protein, 8 aa, synthetic construct
  REGION 1..8: note = Peptide linker
GGSGSGSG

SEQ ID NO: 50 - protein, 6 aa, synthetic construct
  REGION 1..6: note = Peptide linker
GGSGSG

SEQ ID NO: 51 - protein, 4 aa, synthetic construct
  REGION 1..4: note = Peptide linker
GGSG

SEQ ID NO: 52 - protein, 8 aa, synthetic construct
  REGION 1..8: note = Peptide linker
GGSGNGSG

SEQ ID NO: 53 - protein, 8 aa, synthetic construct
  REGION 1..8: note = Peptide linker
GGNGSGSG

SEQ ID NO: 54 - protein, 6 aa, synthetic construct
  REGION 1..6: note = Peptide linker
GGNGSG

SEQ ID NO: 55 - protein, 5 aa, synthetic construct
  REGION 1..5: note = mu CD40 CDR-H1
DYYMA

SEQ ID NO: 56 - protein, 17 aa, synthetic construct
  REGION 1..17: note = mu CD40 CDR-H2
SISYDGSSTYYRDSVKG

SEQ ID NO: 57 - protein, 7 aa, synthetic construct
  REGION 1..7: note = mu CD40 CDR-H3
HSSYFDY

SEQ ID NO: 58 - protein, 11 aa, synthetic construct
  REGION 1..11: note = mu CD40 CDR-L1
RASDSVSTLMH

SEQ ID NO: 59 - protein, 7 aa, synthetic construct
  REGION 1..7: note = mu CD40 CDR-L2
LASHLES

SEQ ID NO: 60 - protein, 9 aa, synthetic construct
  REGION 1..9: note = mu CD40 CDR-L3
QQSWNDPWT

SEQ ID NO: 61 - protein, 226 aa, synthetic construct
  REGION 1..226: note = (P1AE1689) light chain cross VH-Ckappa
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 219 aa, synthetic construct
  REGION 1..219: note = VL (CD40) light chain (charged)
DIVMTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 679 aa, synthetic construct
  REGION 1..679: note = VH (CD40) (VHCH1 charged) Fc knob_PGLALA_(P1AE1689) (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 443 aa, synthetic construct
  REGION 1..443: note = VH (CD40) (VHCH1 charged) Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 65 - protein, 907 aa, synthetic construct
  REGION 1..907: note = VH (CD40) (VHCH1 charged_VH1a (CD40) (VHCH1 charged)-Fc knob_PGLALA_(P1AE1689) (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 66 - protein, 671 aa, synthetic construct
  REGION 1..671: note = VH (CD40) (VHCH1 charged)_VH (CD40) (VHCH1 charged)-Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 67 - protein, 223 aa, synthetic construct
  REGION 1..223: note = 28H1 light chain cross VH-Ckappa
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 676 aa, synthetic construct
  REGION 1..676: note = VH (CD40) (VHCH1 charged) Fc knob_PGLALA_28H1 (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 69 - protein, 213 aa, synthetic construct
  REGION 1..213: note = muCD40 (FGK4.5) light chain
DTVLTQSPALAVSPGERVTISCRASDSVSTLMHWYQQKPGQQPKLLIYLASHLESGVPARFSGSGSGTDFTLTIDPVEADDTATYYCQQSWNDPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 811 aa, synthetic construct
  REGION 1..811: note = muCD40 VHCH1 charged_muCD40 VHCH1 charged-Fc knob_PGLALA_VH (28H1)
EVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 71 - protein, 803 aa, synthetic construct
  REGION 1..803: note = muCD40 VHCH1 charged_muCD40 VHCH1 charged-Fc knob_PGLALA_VL (28H1)
EVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 72 - protein, 810 aa, synthetic construct
  REGION 1..810: note = muCD40 VHCH1 charged_muCD40 VHCH1 charged-Fc knob_PGLALA_VH (DP47)
EVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 73 - protein, 803 aa, synthetic construct
  REGION 1..803: note = muCD40 VHCH1 charged_muCD40 VHCH1 charged-Fc knob_PGLALA_VL (DP47)
EVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 74 - protein, 223 aa, synthetic construct
  REGION 1..223: note = FAP(28H1) light chain cross VH-CL
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 75 - protein, 219 aa, synthetic construct
  REGION 1..219: note = VL (CD40) light chain (with charges)
DIVMTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIKRADAAPTVSIFPPSSRKLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 76 - protein, 669 aa, synthetic construct
  REGION 1..669: note = VH (CD40) (VHCH1 charged) Fc knob_PGLALA FAP(28H1) (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 77 - protein, 437 aa, synthetic construct
  REGION 1..437: note = VH (CD40) (VHCH1 with charges) Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 78 - protein, 116 aa, synthetic construct
  REGION 1..116: note = mu CD40 VH
EVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSS

SEQ ID NO: 79 - protein, 106 aa, synthetic construct
  REGION 1..106: note = mu CD40 VL
DTVLTQSPALAVSPGERVTISCRASDSVSTLMHWYQQKPGQQPKLLIYLASHLESGVPARFSGSGSGTDFTLTIDPVEADDTATYYCQQSWNDPWTFGGGTKLELK

SEQ ID NO: 80 - protein, 119 aa, synthetic construct
  REGION 1..119: note = FAP (212) VH
EVLLQQSGPELVKPGASVKIACKASGYTLTDYNMDWVRQSHGKSLEWIGDIYPNTGGTIYNQKFKGKATLTIDKSSSTAYMDLRSLTSEDTAVYYCTRFRGIHYAMDYWGQGTSVTVSS

SEQ ID NO: 81 - protein, 111 aa, synthetic construct
  REGION 1..111: note = FAP (212) VL
DIVLTQSPVSLAVSLGQRATISCRASESVDNYGLSFINWFQQKPGQPPKLLIYGTSNRGSGVPARFSGSGSGTDFSLNIHPMEEDDTAMYFCQQSNEVPYTFGGGTNLEIK

SEQ ID NO: 82 - DNA, 21 bp, synthetic construct
  misc_feature 1..21: note = fw: primer for mouse Fap
  source 1..21: mol_type = other DNA
gtcacctgatcggcaatttgt

SEQ ID NO: 83 - DNA, 22 bp, synthetic construct
  misc_feature 1..22: note = rev: primer for mouse Fap
  source 1..22: mol_type = other DNA
ccccattctgaaggtcgtagat

SEQ ID NO: 84 - DNA, 22 bp, synthetic construct
  misc_feature 1..22: note = fw: primer for mouse Tbp
  source 1..22: mol_type = other DNA
ccttgtacccttcaccaatgac

SEQ ID NO: 85 - DNA, 21 bp, synthetic construct
  misc_feature 1..21: note = rev: primer for mouse Tbp
  source 1..21: mol_type = other DNA
acagccaagattcacggtaga

(19) United States
(12) Patent Application Publication
(10) Pub. No.: US 2023/0340154 A1
(43) Pub. Date: Oct. 26, 2023

(54) COMBINATION THERAPY WITH FAP-TARGETED CD40 AGONISTS

(71) Applicant: Hoffmann-La Roche Inc., Little Falls, NJ (US)

(72) Inventors: Sara Labiano Alminana, Sarriguren (ES); Pedro Romero, Epalinges (CH); Genrich Tolstonog, Epalinges (CH); Christine Trumpfheller, Zurich (CH); Pablo Umana, Wollerau (CH); Marie Catherine Vozenin, Lausanne (CH)

(73) Assignee: Hoffmann-La Roche Inc., Little Falls, NJ (US)

(21) Appl. No.: 18/318,466

(22) Filed: May 16, 2023

Related U.S. Application Data
(63) Continuation of application No. PCT/EP2021/081610, filed on Nov. 15, 2021.

(30) Foreign Application Priority Data
Nov. 16, 2020 (EP) 20207768.1

(51) Int. Cl. A61P 35/00; C07K 16/40; A61N 5/10; C07K 16/28

(52) U.S. Cl. CPC C07K 16/40; A61N 5/10; A61P 35/00; C07K 16/2878; A61K 2039/505; C07K 2317/31; C07K 2317/75

(57) ABSTRACT

The present invention relates to combination therapies employing FAP-targeted CD40 agonists, in particular bispecific antigen binding molecules comprising at least one antigen binding domain capable of specific binding to Fibroblast Activation Protein (FAP) and at least one antigen binding domain capable of specific binding to CD40, and radiotherapy.

### CROSS REFERENCE TO RELATED APPLICATIONS

This application is a continuation of International Application No. PCT/EP2021/081610 having an international filing date of Nov. 15, 2021, and which claims benefit of priority to European Patent Application No. 20207768.1, filed Nov. 16, 2020; all of which are incorporated herein by reference in their entirety.

### SEQUENCE LISTING

The instant application contains a Sequence Listing which has been submitted electronically in XML file format and is hereby incorporated by reference in its entirety. Said XML copy, created on May 12, 2023, is named P36518-US_SL.xml and is 120,120 bytes in size.

### FIELD OF THE INVENTION

The present invention relates to combination therapies employing FAP-targeted CD40 agonists, in particular bispecific antigen binding molecules comprising at least one antigen binding domain capable of specific binding to Fibroblast Activation Protein (FAP) and at least one antigen binding domain capable of specific binding to CD40, and radiotherapy, and the use of these combination therapies for the treatment of solid tumors and methods of using the combination therapies.

### BACKGROUND

Cancer is one of the leading causes of death worldwide. Despite advances in treatment options, prognosis of patients with advanced cancer remains poor. Consequently, there is a persisting and urgent medical need for optimal therapies to increase survival of cancer patients without causing unacceptable toxicity. Recent results from clinical trials have shown that immune therapies can extend the overall survival of cancer patients and lead to durable responses. Despite these promising results, current immune-based therapies are only effective in a proportion of patients and combination strategies are needed to improve therapeutic benefit.

Radiotherapy (RT) is an important treatment modality for localized tumours. RT typically induces primarily mitotic cell death but also leads to apoptosis [9] and complex effects on the tumour microenvironment, which can facilitate homing of both antigen presenting cells and effector T cells. Sublethal doses of ionizing radiation have been described to directly stimulate major histocompatibility complex (MHC) expression that renders tumor cells more sensitive to detection and lysis by specific T cells.

With an incidence of over 800,000 new cases per year, head and neck cancer is the seventh most common cancer worldwide. Tobacco smoking, alcohol consumption and human papillomavirus (HPV) infection represent the main risk factors in the development of HNSCC (Chow, Head and Neck Cancer, N. Engl. J. Med. 2020, 382(1), 60-72). HPV-related head and neck squamous cell carcinoma (HPV⁺ HNSCC) represents a distinct subgroup of head and neck cancer with improved outcomes compared with HPV-negative HNSCC. This results from a combination of peculiar biologic features of HPV⁺ HNSCC and favorable patient characteristics such as younger age and a minor role for tobacco and alcohol abuse. These patients have shown enhanced and durable benefit from radiotherapy and dose de-escalation trials are currently ongoing (Ventz et al, Clin. Cancer Res. 2019, 25(24), 7281-7286). However, further optimization of treatments by integrating novel combination strategies to enhance therapeutic index and limit toxicity are still required. Amongst these novel options, immuno-modulation appears a promising target as tumor microenvironment characterization in HNSCC biopsies reveals that it is one of the ten most immune-infiltrated cancers. In this regard, the proven immunostimulatory properties of hypofractionated radiotherapy makes this therapeutic modality a good combination partner for immunotherapy (Lhuillier et al., Genome Med. 2019;11(1), 40).

Despite HPV⁺ Head and Neck Squamous Cell Carcinoma (HNSCC) patients have shown good outcome upon radiotherapy, most of them undergo tumor relapses over the time. Thus, further optimization of treatments including novel combination approaches and reduction in the therapy-associated toxicity are still needed.

Among several costimulatory molecules, the TNFR family member CD40 plays a key role in triggering immune responses by inducing maturation, survival, antigen presentation, cytokine production, and expression of costimulatory molecules of antigen-presenting cells (APCs), which then drive antigen-specific T cell responses by proinflammatory cytokines. CD40 regulates immune responses against infections, tumors and self-antigens and its expression has been demonstrated on the surface of APCs such as B cells, dendritic cells (DCs), monocytes, and macrophages as well as platelets, and cells of non-hematopoietic origin such as myofibroblasts, fibroblasts, epithelial, and endothelial cells (Elgueta R. et al., Immunol Rev. 2009; 229(1):152-72). The CD40 ligand CD40L is expressed on activated CD4⁺ helper T cells, platelets, monocytic cells, natural killer cell, mast cells, and basophils (Carbone E. et. al., J Exp Med. 1997;185(12): 2053-2060, or Elgueta R. et al., Immunol Rev. 2009;229(1):152-72). Expression of CD40 and CD40L is strongly upregulated in response to various immune stimulatory signals and CD40-CD40L interaction between APCs and CD4⁺ T cells contributes to increased APC activation and antigen-specific CD8⁺ T cell responses (Bevan MJ., Nat Rev Immunol. 2014;4(8):595-602). Similar immune stimulatory results were observed by using CD40 agonistic antibodies (Vonderheide RH and Glennie MJ., Clin Cancer Res. 2013;19(5):1035-43).

Employing agonistic anti-CD40 antibodies, which can mimic this so-called licensing of APCs, therefore represents a promising strategy in the context of cancer immunotherapy. In particular, the ability of anti-CD40 molecules to activate DCs and subsequently increase (cross)priming of tumor-specific T cells has made them interesting for clinical development. A number of agonistic anti-CD40 antibodies have been developed over the last years: Chi Lob 7/4 (CD40 agonistic IgG1 chimeric mAb; Cancer Research UK; Chowdhury F. et al., Cancer Immunol Res. 2013;2:229-40), ADC1013 (fully human, CD40 agonistic IgG1 antibody; Alligator Bioscience and Johnson & Johnson; Mangsbo SM. et al., Clin Cancer Res. 2015 Mar 1;21(5):1115-26), APX-005 (fully humanized, CD40 agonistic IgG1 mAb; Apexigen; Bjorck P. et al. J Immunother Cancer. 2015; 3(Suppl 2): P198), SEA-CD40 (CD40 agonistic IgG1 chimeric mAb; Seattle Genetics; Gardai SJ. et al. AACR 106th Annual Meeting 2015; April 18-22, abstract 2472), as well as RO7009789 (fully human, CD40 super agonistic IgG2 mAb) are or have been investigated in clinical phase I studies, and dacetuzumab (CD40 partial agonistic IgG1 chimeric mAb; Seattle Genetics; Khubchandani S. et al., Curr Opin Investig Drugs. 2009;10, 579-87) has been investigated in a clinical phase II study. Eligible patients for these studies had solid tumors, classical Hodgkin lymphoma (HL), diffuse large B-cell lymphoma (DLBCL), or indolent lymphoma (including follicular lymphoma). Although the clinical development of agonistic CD40 antibodies has provided promising initial results, major challenges of anti-CD40 agonist treatments were revealed. Across different clinical studies, dose-limiting side effects such as cytokine release syndrome and hepatotoxicity, and thromboembolic events were observed, so that with the dose schedules and routes of administration used only a limited clinical efficacy and a local administration of the antibody may be possible. The lack of single agent responses occurs in part due to severe on target/off tumor effects caused by broad CD40 expression, which results in dose limiting toxicity (e.g. cytokine release syndrome). The development of an agonistic CD40 antibody that specifically activates APCs when CD40 is cross-linked by a tumor-specific target may reduce side effects and decrease dose limitations, offering new therapeutic options with the potential to generate an efficient long lasting anti-cancer immunity.

Bispecific antigen binding molecules capable of specific binding to CD40 and Fibroblast Activation Protein (FAP) are described in WO 2018/185045 A1, WO 2020/070041 A1 or WO 2020/070035 A1. These molecules combine a moiety capable of binding to FAP with a moiety capable of agonistic binding to CD40, wherein the activation of APCs through CD40 is provided by cross-linking through FAP expressed on tumor stroma cells and potentially also through FAP intermediately expressed in secondary lymphoid tissues. In contrast to bispecific antigen binding molecules capable of specific binding to CD40 and to immune checkpoint receptors on activated T cells, such as CTLA-4 or PD-1, targeting to a tumor target such as FAP enables CD40-mediated APC activation mainly in the tumor stroma and tumor-draining lymph nodes where fibroblasts express increased levels of FAP compared to other tissues. The FAP-targeted agonistic CD40-antigen binding molecules may thus be able to trigger the CD40 receptor not only effectively, but also very selectively at the desired site while overcoming the need for FcγR cross-linking thereby reducing side effects.

We herein describe a novel combination therapy employing FAP-targeted CD40 agonists, in particular bispecific antigen binding molecules comprising at least one antigen binding domain capable of specific binding to Fibroblast Activation Protein (FAP) and at least one antigen binding domain capable of specific binding to CD40 (FAP-CD40 bispecific antibody) in combination with radiotherapy, in particular local hypo-fractionated radiation therapy.

### SUMMARY OF THE INVENTION

The present invention relates to bispecific agonistic CD40-antigen binding molecules, in particular bispecific agonistic CD40-antigen binding molecules comprising at least one antigen binding domain capable of specific binding to a tumor-associated antigen, and their use in combination with radiotherapy, in particular to their use in a method for treating a solid tumor in an individual, in particular a human. It has been found that the combination therapy described herein is more effective in inhibiting tumor growth and eliminating tumor cells than treatment with bispecific agonistic CD40-antigen binding molecules alone or radiotherapy alone.

Thus, provided a bispecific agonistic CD40-antigen binding molecule for use in treating a solid tumor in an individual, wherein the bispecific agonistic CD40-antigen binding molecule is used in combination with radiotherapy and wherein the bispecific agonistic CD40-antigen binding molecule comprises at least one antigen binding domain capable of specific binding to a tumor-associated antigen. In particular, the tumor-associated antigen is Fibroblast Activation Protein (FAP).

In some aspects, the bispecific agonistic CD40-antigen binding molecule comprises at least one antigen binding domain capable of specific binding to CD40 and at least one antigen binding domain capable of specific binding to Fibroblast Activation Protein (FAP).

In some aspects, the bispecific agonistic CD40-antigen binding molecule is for simultaneous or sequential administration with the radiotherapy. In some aspects, the bispecific agonistic CD40-antigen binding molecule is for administration after the radiotherapy. In some aspects, the bispecific agonistic CD40-antigen binding molecule is for single administration after the radiation. In some aspects, the bispecific agonistic CD40-antigen binding molecule is for single administration one day after the radiation. In some aspects, the bispecific agonistic CD40-antigen binding molecule is for administration one day after the radiation is finished. In some aspects, the radiotherapy comprises local radiotherapy. In some aspects, the local radiotherapy is selected from external beam radiation therapy or brachytherapy. In one aspect, the radiotherapy is external beam radiation therapy. In another aspect, the radiotherapy is brachytherapy. In some aspects, the radiotherapy comprises local hypofractionated radiation at a dose in the range of 1.8 to 20 Gy, particularly in the range of 5 to 20 Gy. In some aspects, the the radiotherapy comprises local hypofractionated radiation at a dose of 6 Gy, preferably 2 × 6 Gy. In some aspects, the radiotherapy comprises local hypofractionated radiation at a dose in the range of 1.8 to 2.2 Gy, preferably 2 Gy.

In some aspects, provided is a bispecific agonistic CD40-antigen binding molecule for use in treating a solid tumor in an individual, wherein the bispecific agonistic CD40-antigen binding molecule comprises at least one antigen binding domain capable of specific binding to a tumor-associated antigen and wherein the solid tumor is selected from the group consisting of head and neck cancer, melanoma, lung cancer, kidney cancer, breast cancer, colon cancer, ovarian cancer, cervical cancer, pancreatic cancer, liver cancer, prostate cancer, bladder cancer, gastric cancer and glioblastoma. In some aspects, the tumor-associated antigen is FAP and the tumor is characterized by a FAP-expressing stroma. In some aspects, the solid tumor to be treated is head and neck cancer or lung cancer. In particular, the solid tumor is head and neck cancer, more particularly HPV⁺ Head and Neck Squamous Cell Carcinoma (HNSCC). In another particular aspect, the solid tumor is lung cancer, in particular non-small cell lung cancer (NSCLC).

In some aspects, the bispecific agonistic CD40-antigen binding molecule for use in treating a solid tumor in an individual is an antigen binding molecule comprising an IgG Fc domain, specifically an IgG1 Fc domain or an IgG4 Fc domain, and wherein the Fc domain comprises one or more amino acid substitutions that reduce the binding affinity of the antibody to an Fc receptor and/or effector function. In particular, the bispecific agonistic CD40-antigen binding molecule comprises a Fc domain of human IgG1 subclass with the amino acid substitutions L234A, L235A and P329G (numbering according to Kabat EU index).

In some aspects, the bispecific agonistic CD40-antigen binding molecule for use in treating a solid tumor in an individual is a bispecific agonistic CD40-antigen binding molecule comprising at least one antigen binding domain capable of specific binding to CD40 and at least one antigen binding domain capable of specific binding to Fibroblast Activation Protein (FAP), wherein at least one antigen binding domain capable of specific binding to CD40 comprises a heavy chain variable region (V_{H}CD40) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}CD40) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6. In particular, the bispecific agonistic CD40-antigen binding molecule comprises at least one antigen binding domain capable of specific binding to CD40 comprising a VH comprising the amino acid sequence of SEQ ID NO:7 and a VL comprising the amino acid sequence of SEQ ID NO:8.

In further aspects, the bispecific agonistic CD40-antigen binding molecule comprises at least one antigen binding domain capable of specific binding to FAP comprising
(a) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or
(b) a heavy chain variable region (V_{H}FAP) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a light chain variable region (V_{L}FAP) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(c) a heavy chain variable region (V_{H}FAP) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}FAP) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In some aspects, the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:9, (ii) CDR-H2 comprising the amino acid sequence of SEQ ID NO: 10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14. In another aspects, the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a light chain variable region (V_{L}FAP) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22. In yet another aspects, the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}FAP) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In some aspects, the bispecific agonistic CD40-antigen binding molecule comprises at least one antigen binding domain capable of specific binding to FAP comprising
(a) a heavy chain variable region (V_{H}FAP) comprising the amino acid sequence of SEQ ID NO:15 and a light chain variable region (V_{L}FAP) comprising the amino acid sequence of SEQ ID NO: 16,
(b) a heavy chain variable region (V_{H}FAP) comprising the amino acid sequence of SEQ ID NO:23 and a light chain variable region (V_{L}FAP) comprising the amino acid sequence of SEQ ID NO:24, or
(c) a heavy chain variable region (V_{H}FAP) comprising the amino acid sequence of SEQ ID NO:31 and a light chain variable region (V_{L}FAP) comprising the amino acid sequence of SEQ ID NO:32.

In particular aspects, the bispecific agonistic CD40-antigen binding molecule comprises at least one antigen binding domain capable of specific binding to FAP comprising a heavy chain variable region (V_{H}FAP) comprising the amino acid sequence of SEQ ID NO:15 and a light chain variable region (V_{L}FAP) comprising the amino acid sequence of SEQ ID NO:16.

Thus, in particular aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and (ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:16.

In another aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and (ii) at least one antigen binding domain capable of specific binding to FAP, comprising a heavy chain variable region (V_{H}FAP) comprising an amino acid sequence of SEQ ID NO:23 and a light chain variable region (V_{L}FAP) comprising an amino acid sequence of SEQ ID NO:24. In other aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and (ii) at least one antigen binding domain capable of specific binding to FAP, comprising a heavy chain variable region (V_{H}FAP) comprising an amino acid sequence of SEQ ID NO:31 and a light chain variable region (V_{L}FAP) comprising an amino acid sequence of SEQ ID NO:32.

In some aspects, the bispecific agonistic CD40-antigen binding molecule comprises
a) at least two Fab fragments capable of specific binding to CD40 fused at its C-terminus to the N-terminus of a Fc region, and
(b) one antigen binding domain capable of specific binding to FAP fused at its N-terminus to the C-terminus of the Fc region.

In some aspects, the bispecific agonistic CD40-antigen binding molecule comprises
a) at least two Fab fragments capable of specific binding to CD40 fused at its C-terminus to the N-terminus of a Fc region, and
(b) a cross-fab fragment capable of specific binding to FAP fused to the C-terminus of the Fc region.

In particular aspects, the bispecific agonistic CD40-antigen binding molecule comprises a cross-fab fragment capable of specific binding to FAP, wherein the VH-Ckappa chain of the cross-fab fragment is fused to the C-terminus of the Fc region. In particular aspects, the bispecific agonistic CD40-antigen binding molecule comprises two Fab fragments capable of specific binding to CD40 fused at its C-terminus to the N-terminus of a Fc region. In particular aspects, the bispecific agonistic CD40-antigen binding molecule is characterized by bivalent binding to CD40 and monovalent binding to FAP.

In another aspects, the bispecific agonistic CD40-antigen binding molecule is characterized by tetravalent or trivalent binding to CD40 and monovalent binding to FAP. In some aspects, the bispecific agonistic CD40-antigen binding molecule is characterized by tetravalent binding to CD40 and monovalent binding to FAP. In some aspects, the bispecific agonistic CD40-antigen binding molecule comprises four Fab fragments capable of specific binding to CD40, wherein each two Fab fragments are fused to each other and fused at its C-terminus to the N-terminus of a Fc region.

In some aspects, the bispecific agonistic CD40-antigen binding molecule is for use of treating a solid tumor, wherein (i) the individual has increased survival when treated with a therapeutically effective amount of the bispecific agonistic CD40-antigen binding molecule in combination with radiotherapy compared with an individual who received the bispecific agonistic CD40-antigen binding molecule as monotherapy or who received radiotherapy as monotherapy, or (ii) wherein the size of the solid tumor in the individual is reduced by more than the additive amount and by which the size is reduced by treatment with the bispecific agonistic CD40-antigen binding molecule used as monotherapy and the radiotherapy used as monotherapy.

In a further aspect, provided is a composition comprising a bispecific agonistic CD40-antigen binding molecule in a pharmaceutically effective amount for use in the treatment of a solid tumor, wherein the treatment comprises the combination with radiotherapy and the bispecific agonistic CD40-antigen binding molecule comprises at least one antigen binding domain capable of specific binding to a tumor-associated antigen. In particular, the tumor associated antigen is FAP.

In some aspects, provided is a pharmaceutical composition comprising a bispecific agonistic CD40-antigen binding molecule in a pharmaceutically effective amount for use in treating a solid tumor in an individual in combination with radiotherapy, wherein the bispecific agonistic CD40-antigen binding molecule comprises at least one antigen binding domain capable of specific binding to a tumor-associated antigen. In particular, the tumor-associated antigen is FAP.

In another aspect, provided is the use of a bispecific agonistic CD40-antigen binding molecule in the manufacture of a medicament for treating a solid tumor in an individual, wherein the bispecific agonistic CD40-antigen binding molecule is for combination with radiotherapy and comprises at least one antigen binding domain capable of specific binding to a tumor-associated antigen.

In another aspect, provided is a method for treating a solid tumor in an individual comprising administering to the subject an effective amount of a bispecific agonistic CD40-antigen binding molecule and an effective amount of radiotherapy, wherein the bispecific agonistic CD40-antigen binding molecule comprises at least one antigen binding domain capable of specific binding to a tumor-associated antigen.

In all of these aspects, the individual is preferably a human.

### BRIEF DESCRIPTION OF THE DRAWINGS

FIGS. 1A to D show schematic representations of the antigen binding molecules which specifically bind to human CD40. FIG. 1A shows a schematic representation of a bispecific FAP-CD40 antibody in a 4+1 format consisting of four CD40 binding moieties combined with one FAP binding moiety as crossover fab fragment, wherein the VL-CH1 chain is fused at the C-terminus of the Fc knob chain (tetravalent binding for CD40 and monovalent binding for FAP). In a control molecule with the same structure, the FAP binding moiety is replaced by the VH and VL domains of the germline control DP47.FIG. 1B shows a schematic representation of a bispecific FAP-CD40 antibody in a 4+1 format consisting of four CD40 binding moieties combined with one FAP binding moiety as VH and VL domains, wherein the VH domain is fused at its N-terminus to the C-terminus of the Fc knob chain and the VL domain is fused at its N-terminus to the C-terminus of the Fc hole chain (tetravalent binding for CD40 and monovalent binding for FAP). In a control molecule, the FAP binding moiety is replaced by the VH and VL domains of the germline control DP47. FIG. 1C shows a schematic representation of a bispecific CD40 antibody with an Fc domain the amino acid substitutions L234A, L235A and P329G (“P329G LALA”, EU numbering) in the IgG heavy chains (bivalent binding for CD40). FIG. 1D shows a schematic representation of a bispecific FAP-CD40 antibody in a 2+1 format consisting of two CD40 binding moieties combined with one FAP binding moiety as crossover fab fragment, wherein the VL-CH1 chain is fused at the C-terminus of the Fc knob chain (bivalent for CD40 and monovalent for FAP). The black point symbolizes knob-into-hole mutations. In a control molecule, the FAP binding moiety is replaced by the VH and VL domains of the germline control DP47.

FIGS. 2A to 2H show the efficacy of a bispecific FAP-CD40 antibody (P1AD9139, FIG. 1B), either alone or in combination with radiotherapy (RT), in the orthotopic head and neck tumor mouse model (mEERL95) with FAP-expressing stroma. FIG. 2A is a scheme showing the treatment schedule applied to established submental mEERL95 mouse tumors. FIG. 2B shows the survival of mEERL95 tumor-bearing mice subjected to the indicated treatments including the control antibody DP47-CD40 ((P1AE2425) as Log-rank test compared to untreated group (p=0.233). Radiotherapy alone (2 × 6 Gy) did not much increase the probabilility of survival (p=0.001) as well as therapy with FAP-CD40 alone (p=0.024), whereas the combination of RT (2 × 6 Gy) with FAP-CD40 treatment led to a high probability of survival (p=0.0006). Log-rank test was used for statistical analysis comparing the indicated group with untreated mice. n=5-6 mice/group. FIGS. 2C to 2H show the mEERL95 tumor growth curves (represented as tumor volumes) undergoing the indicated groups of treatment. The number of mice that rejected the tumor is indicated for each group (FIG. 2C: untreated mice, FIG. 2D: mice treated with control DP47-CD40, FIG. 2E: mice treated with FAP-CD40, FIG. 2F: mice treated with RT (2 x 6 Gy), FIG. 2G: mice treated with RT (2 × 6 Gy) and DP47-CD40, FIG. 2H: mice treated with RT (2 × 6 Gy) and FAP-CD40). FIGS. 2I to 2K show the mEERL95 tumor growth curves (represented as tumor volumes) of a further experiment, wherein a single dose (FIG. 2K) or two doses of anti-FAP-CD40 (FIG. 2L) were combined with hypofractionated radiotherapy and compared with each other, no treatment (FIG. 2I) and radiotherapy alone (FIG. 2J).

FIGS. 3A to 3G relate to a second experiment comparing the bispecific FAP-CD40 antibody therapy to therapy with a conventional anti-CD40 agonistic antibody. mEERL95 tumor growth curves on mice subjected to the indicated treatments including the non-targeted anti-CD40 (FGK4.5) mAb and the respective IgG-matched control antibody (IgG Ctr). FIG. 3A: mice treated with IgG Ctr, FIG. 3B: mice treated with anti-CD40 mAb, FIG. 3C: mice treated with anti-CD40 mAb and RT (2 × 6 Gy), FIG. 3D: mice treated with IgG Ctr and RT (2 × 6 Gy), FIG. 3E: mice treated with FAP-CD40, FIG. 3F: mice treated with RT (2 × 6 Gy) and FAP-CD40). One-way ANOVA test was used for the differences in the body weight of mice upon the indicated treatments compared to the control group. n=5-10 mice/group. The percentage of body weight gain/loss 72 h upon the indicated treatments is shown in FIG. 3G.

FIGS. 4A to 4K show the results of an experiment studying the effect of FAP expression by fibroblasts at the tumor bed on the efficiency of FAP-CD40. In FIG. 4A, a comparison of DAPI, FAP and αSMA immunofluorescence staining in TC-1 tumor cells and mEERL95 submental tumor cells 10 days post-inoculation is shown. The FAP mRNA levels from mEERL95 and TC-1 submental tumors measured by qPCR are shown in FIG. 4B, whereas FIG. 4C shows the CD40 mRNA levels from mEERL95 and TC-1 submental tumors measured by qPCR. FIGS. 4D to 4G show the TC-1 tumor growth curves (represented as tumor volumes) of mice subjected to the indicated treatments following the schedule as in FIG. 2A. The number of mice that rejected the tumor is indicated for each group. FIG. 4D: untreated mice, FIG. 4E: mice treated with FAP-CD40, FIG. 4F: mice treated with RT (2 × 6 Gy), FIG. 4G: mice treated with RT (2 × 6 Gy) and FAP-CD40. FIG. 4H illustrates how FAP-CD40 bispecific accumulates in TC-1 tumors and in mEERL95 tumors. Shown is the quantification of the fluorescence obtained from the tumors. FI: Fluorescence intensity. n=2-3 mice/group. The results of IHC of FAP performed in cervical lymph nodes 10 days upon mEERL95 and TC-1 cells inoculation in the submental region are shown in FIG. 4I. The percentage of FAP positive cells (FIG. 4J) and MFI of FAP (FIG. 4K) measured by flow cytometry on FRC (gp38+CD31-) from cervical lymph nodes of naïve, mEERL95 and TC-1 tumor bearing mice 10 days post-inoculation is also illustrated. MFI: mean of fluorescence. Mann-Whitney test; ***: p<0.001; n=5-10 mice/group.

The results of a tumor rechallenge experiment are shown in FIGS. 5A to 5E. FIG. 5A is a scheme showing the schedule of sequential tumor rechallenges on long-term responders to combination treatment with RT (2 × 6 Gy) and anti-FAP-CD40. The tumor growth curves after rechallenge with mEERL95 tumor cells (FIG. 5B) and TC-1 tumor cells (FIG. 5C) upon sequential re-challenge in the right (1×10⁶ cells) and left flanks (1×10⁵ cells) are shown, respectively. The generation of tumor-specific memory is observed with the combination treatment. FIG. 5D shows the percentage of tetramer positive CD8 T cells from PMBC of cured mice restimulated with the E7 peptide for 8 days. FIG. 5E shows the percentage of tetramer positive CD8 T cells on TILs from mEERL95-bearing mice obtained 6 days after the indicated treatments. **: p<0.01, ***: p<0.001; Kruskal Wallis test was used for the statistical analyses. mean±SEM.; n=5-8 mice/group.

FIGS. 6A to 6F relate to a further experiment using transgenic mice expressing the human CD40 receptor (huCD40 Tg mice) and anti-human FAP-CD40 in 2+1 format (P1AE2302). FIG. 6A is a scheme showing the schedule of treatment applied to mEERL95 tumor-bearing huCD40 Tg mice. FIG. 6B summarizes the percentage of survival of mEERL95-bearing huCD40 transgenic mice upon the indicated treatments. The number of mice that rejected the tumor is indicated on each group. Log-rank test; ns: non-significant, **: p<0.01, ***: p<0.001; n=10 mice/group. The tumor growth curves of MEERL95-bearing huCD40 transgenic mice upon the indicated treatments are shown for untreated mice (FIG. 6C), mice treated with anti-human FAP-huCD40 (FIG. 6D), mice treated with RT (2 × 6 Gy) only (FIG. 6E) and for mice treated with the combination of RT (2 × 6 Gy) and anti-human FAP-huCD40 (FIG. 6F).

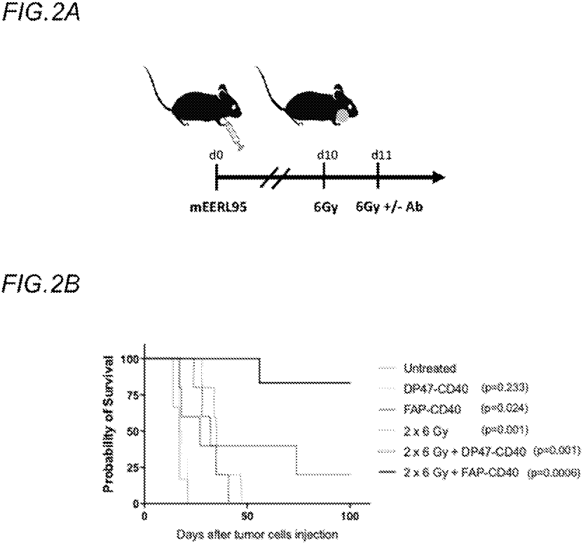

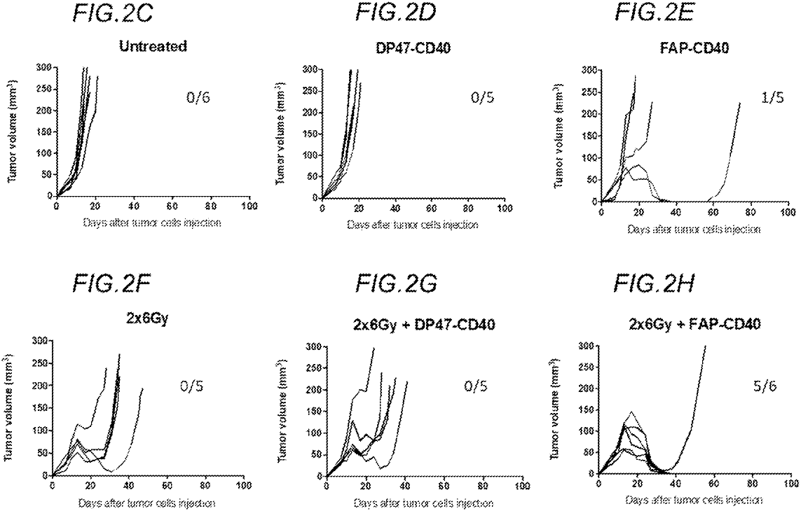

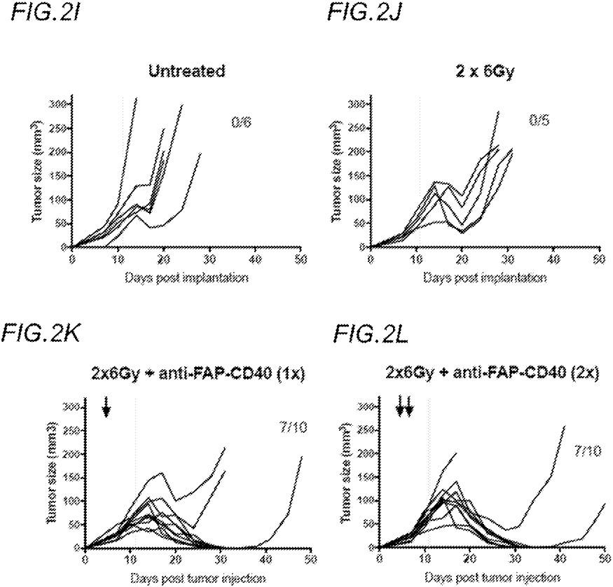

FIGS. 7A to 7H show the analysis of the cellular and molecular immune components associated with the anti-tumor effect of the treatments of the experiment described in FIG. 2A. Flow cytometry analysis of the indicated immune populations in tumors at day 18 post mEERL95 tumor cell inoculation (i.e. 7 days post FAP-CD40 treatment). The number of immune cells is shown for CD45+ cells (FIG. 7A), CD8 T cells (FIG. 7B), regulatory T cells (Tregs, FIG. 7C), CD4 T cells (FIG. 7F), dendritic cells (FIG. 7G) and NK cells (FIG. 7H) per mg of tumor, respectively. The ratio of CD8/Tregs and the ratio of CD8/macrophages is shown in FIGS. 7D and 7E, respectively.

A comparison between the group of untreated mice and mice treated with the combination of RT and FAP-CD40 of the production of IFNγ, TNFα and Granzyme B by tumor-infiltrating CD8 lymphocytes upon 16 h restimulation with mEERL95 tumor cells as measured by intracellular FACS staining is shown in FIGS. 8A, 8B and 8C, respectively. FIGS. 8D, 8E and 8F show the production of IFNγ, TNFα and Granzyme B by tumor-infiltrating CD4 lymphocytes, respectively. FIG. 8G shows the frequency of Ki67 positive cells and FIG. 8H shows the PD1 expression within the CD8 T cell population infiltrating the tumors upon different treatments. In FIG. 8I is shown the frequency of CD62L CD44 double positive cells (memory phenotype) within the CD8 T cell population from cervical lymph nodes.

FIGS. 9A, 9B and 9C show the levels of chemokines CXCL9 and CXCL10 and cytokines (INFγ) in the tumor microenvironment at day 8 post treatment with the different therapies measured by multiplex, respectively. FIGS. 9D and 9E show the percentage of dendritic cells (CD11c+ MHCII+) expressing the indicated surface maturation markers CD80 and CD86, respectively, from mEERL95 tumors at day 4 following the indicated treatments following the indicated treatments. Mann-Whitney test, One-way ANOVA or Kruskal Wallis test were used for the statistical analyses. mean±SEM. *: p<0.05, **: p<0.01, ***: p<0.001. n=3-6 samples/group. FIG. 9G shows the quantification of the FAP+ area and location upon the different conditions in mEERL95 tumors 7 days upon the indicated treatments. FIG. 9H is a heatmap of gene signature associated to DC maturation differentially expressed in the tumor microenvironment four days after the indicated treatments..

In FIGS. 10A and 10B mEERL95 tumor-bearing mice were depleted of T lymphocytes using an anti-CD8β, an anti-CD4 or the respective isotype control antibody starting 48 h prior to the combination therapy administration. It is shown that the depletion of CD8 T lymphocytes resulted in an abrogation of the curative effect observed upon the combination therapy. Mice depleted of CD4 T lymphocytes underwent tumor relapses, indicating an essential role of CD4 T cells in the formation of durable anti-tumor responses. FIGS. 10A to 10D show the mEERL95 tumor growth curves (represented as tumor volumes) upon no treatment (FIG. 10A) and treatment with the combination of RT (2 × 6 Gy) and FAP-CD40 when not depleted (FIG. 10B) and when depleted of CD8 (FIG. 10C) or CD4 T cells (FIG. 10D). FIG. 10E shows the survival of mice that underwent the combination treatment including the indicated depleting antibodies. Log-rank test; ns: non-significant, **: p<0.01, ***: p<0.001; n=7 mice/group.

The combination therapy in the absence of cross-priming DCs was studied in FIGS. 10F to 10I. FIGS. 10F and 10G show the mEERL95 tumor growth curves in Batf3-/-mice (which lack cDC1) that are untreated or treated with the combination of RT (2 × 6 Gy) and FAP-CD40. FIGS. 10H and 10I show the mEERL95 tumor growth curves in tumor-bearing wild-type mice that are untreated or treated with the combination of RT (2 × 6 Gy) and FAP-CD40, respectively, indicating the requirement of cross-priming DCs for the FAP-CD40-mediated robust protective response.

The mEERL95 tumor growth curves in mice untreated or treated with the combination therapy of RT (2 × 6 Gy) and FAP-CD40 and additionally an IL-12 blocking antibody or the respective isotype-matched control are shown in FIGS. 11A, 11B and 11C, respectively. The number of mice and the number of mice that rejected the tumor is indicated in each treatment group. The corresponding levels of chemokines measured by multiplex in the serum of mice in each of these treatment groups 21 days after tumor injection are shown in FIG. 11D (CXCL9 levels), FIG. 11E (CXCL10 levels), FIG. 11F (CCL4 levels) and FIG. 11G (CCL22 levels). One-way ANOVA or Kruskal Wallis test were used for the statistical analyses. mean±SEM. ^{∗}: p<0.05, ^{∗∗}: p<0.01. n=5-9 samples/group.

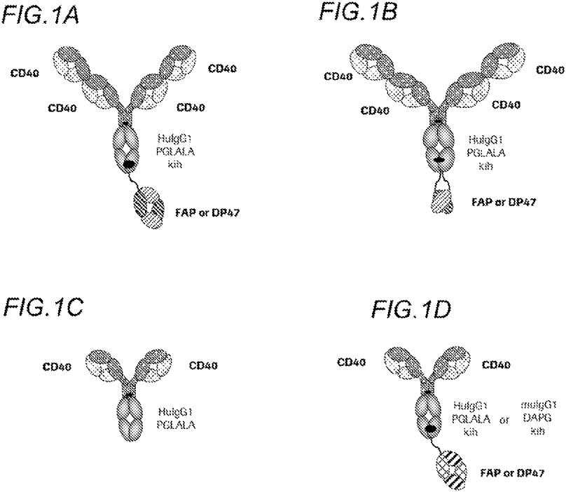

FIGS. 12A to 2C show the efficacy of a bispecific FAP-CD40 antibody (P1AD9139, FIG. 1B), either alone or in combination with radiotherapy (RT), in the orthotopic SV2-OVA lung tumor model with FAP-expressing stroma. FIG. 12A is a scheme showing the treatment schedule applied to established SV2-OVA mouse tumors. FIG. 12B shows the reduction of normal lung volume in % for each treatment group. A decrease in lung volume was observed in the group of mice (n=7) that received radiotherapy (RT, 2 × 6 Gy) much later than in the untreated animals (n=7). For the group receiving both RT (2 × 6 Gy) and FAP-CD40 treatment (2 times at day 14 and day 18) a decrease of lung volume was only observed after day 41, much later as with RT alone. FIG. 12C shows the survival of SV2-OVA tumor-bearing mice subjected to the indicated treatments including the control antibody DP47-CD40 ((P1AE2425) as Log-rank test compared to untreated group. Radiotherapy alone (2 × 6 Gy) did increase the probabilility of survival, however the combination of RT (2 × 6 Gy) with FAP-CD40 treatment led to a much higher probability of survival.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

As used herein, the term “antigen binding domain capable of specific binding to a tumor-associated antigen” or “moiety capable of specific binding to tumor-associated anitgen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the CD40 agonist) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565). In particular, the target cell antigen is FAP.

In relation to an antibody or fragment thereof, the term “antigen binding domain capable of specific binding to an antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a target cell antigen” can also be a Fab fragment or a cross-Fab fragment.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells. A bispecific antigen binding molecule as described herein can also form part of a multispecific antibody.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules can be monovalent for a certain antigenic determinant, meaning that they have only one binding site for said antigenic determinant or they can be bivalent, trivalent or tetravalent for a certain antigenic determinant, meaning that they have two binding sites, three binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or µ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′ -SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J.S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain- like Ig fold. Loops corresponding to CDRs of antibodies can be substituted with heterologous sequence to confer different binding properties. CTLA-4 molecules engineered to have different binding specificities are also known as Evibodies (e.g. US7166697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), US7250297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556 - 1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435- 444 (2005), US20080139791, WO2005056764 and US6818418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges - examples of microproteins include KalataBI and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more. An “antigen binding molecule that does not bind to the same epitope” as a reference molecule refers to an antigen binding molecule that does not block binding of the reference molecule to its antigen in a competition assay by 50% or more, and conversely, the reference molecule does not block binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, a molecule that binds to the antigen has a dissociation constant (Kd) of ≤ 1 µM, ≤ 100 nM, ≤ 10 nM, ≤ 1 nM, ≤ 0.1 nM, ≤ 0.01 nM, or ≤ 0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of noncovalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “affinity matured” antibody refers to an antibody with one or more alterations in one or more hypervariable regions (HVRs or CDRs), compared to a parent antibody which does not possess such alterations, such alterations resulting in an improvement in the affinity of the antibody for antigen.

A “tumor-associated antigen” as used herein refers to an antigenic determinant presented on the surface of a target cell, in particular a target cell in a tumor such as a cancer cell or a cell of the tumor stroma. In particular, the tumor-associated antigen is Fibroblast Activation Protein (FAP).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:34), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NO: 35. The amino acid sequence of mouse FAP is shown in UniProt accession no. P97321 (version 126, SEQ ID NO:36), or NCBI RefSeq NP_032012.1. The extracellular domain (ECD) of mouse FAP extends from amino acid position 26 to 761. SEQ ID NO: 37 shows the amino acid sequence of a His-tagged mouse FAP ECD. SEQ ID NO: 38 shows the amino acid sequence of a His-tagged cynomolgus FAP ECD. Preferably, an anti-FAP binding molecule of the invention binds to the extracellular domain of FAP.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and µ respectively..

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “Fc domain” or “Fe region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in US 5,731,168; US 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J.G. and Anderson, C.L., J. Leukoc. Biol. 49 (1991) 511-524). FcRs are defined by their specificity for immunoglobulin isotypes: Fc receptors for IgG antibodies are referred to as FcyR. Fc receptor binding is described e.g. in Ravetch, J.V. and Kinet, J.P., Annu. Rev. Immunol. 9 (1991) 457-492, Capel, P.J., et al., Immunomethods 4 (1994) 25-34; de Haas, M., et al., J. Lab. Clin. Med. 126 (1995) 330-341; and Gessner, J.E., et al., Ann. Hematol. 76 (1998) 231-248.

Cross-linking of receptors for the Fc-region of IgG antibodies (FcyR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K.L., et al., Eur. J. Immunol. 29 (1999) 2613-2624).
-FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On β-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R.L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “CD40”, as used herein, refers to any native CD40 from any vertebrate source, including mammals such as primates (e.g. humans) and rodents (e.g., mice and rats), unless otherwise indicated. The term encompasses “full-length,” unprocessed CD40 as well as any form of CD40 that results from processing in the cell. The term also encompasses naturally occurring variants of CD40, e.g., splice variants or allelic variants. The amino acid sequence of an exemplary human CD40 is shown in SEQ ID NO:33 (Uniprot P25942, version 200) and the amino acid sequence of an exemplary mouse CD40 is shown in SEQ ID NO: 39 (Uniprot P27512, version 160). The CD40 antigen is a 50 kDa cell surface glycoprotein which belongs to the Tumor Necrosis Factor Receptor (TNF-R) family. (Stamenkovic et al. (1989), EMBO J. 8: 1403-10). CD40 is expressed in many normal and tumor cell types, including B lymphocytes, dendritic cells, monocytes, macrophages, thymus epithelium, endothelial cells, fibroblasts, and smooth muscle cells. CD40 is expressed in all B-lymphomas and in 70% of all solid tumors and is up-regulated in antigen presenting cells (APCs) by maturation signals, such as IFN-gamma and GM-CSF. CD40 activation also induces differentiation of monocytes into functional dendritic cells (DCs) and enhances cytolytic activity of NK cells through APC-CD40 induced cytokines. Thus CD40 plays an essential role in the initiation and enhancement of immune responses by inducing maturation of APCs, secretion of helper cytokines, upregulation of costimulatory molecules, and enhancement of effector functions.

The term “CD40 agonist” as used herein includes any moiety that agonizes the CD40/CD40L interaction. CD40 as used in this context refers preferably to human CD40, thus the CD40 agonist is preferably an agonist of human CD40. Typically, the moiety will be an agonistic CD40 antibody or antibody fragment.

The terms “anti-CD40 antibody”, “anti-CD40”, “CD40 antibody and “an antibody that specifically binds to CD40” refer to an antibody that is capable of binding CD40 with sufficient affinity such that the antibody is useful as a diagnostic and/or therapeutic agent in targeting CD40. In one aspect, the extent of binding of an anti-CD40 antibody to an unrelated, non-CD40 protein is less than about 10% of the binding of the antibody to CD40 as measured, e.g., by a radioimmunoassay (RIA) or flow cytometry (FACS). In certain embodiments, an antibody that binds to CD40 has a dissociation constant (K_{D}) of ≤ 1 µM, < 100 nM, ≤ 10 nM, ≤ 1 nM, ≤ 0.1 nM, ≤ 0.01 nM, or ≤ 0.001 nM (e.g. 10⁻⁶ M or less, e.g. from 10⁻⁶⁸ M to 10⁻¹³ M, e.g., from 10⁻⁸ M to 10⁻¹⁰ M).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO:40), GGGGSGGGGS (SEQ ID NO:41), SGGGGSGGGG (SEQ ID NO:42) and GGGGSGGGGSGGGG (SEQ ID NO:43), but also include the sequences GSPGSSSSGS (SEQ ID NO:44), (G4S)₃ (SEQ ID NO:45), (G4S)₄ (SEQ ID NO:46), GSGSGSGS (SEQ ID NO:47), GSGSGNGS (SEQ ID NO:48), GGSGSGSG (SEQ ID NO:49), GGSGSG (SEQ ID NO:50), GGSG (SEQ ID NO:51), GGSGNGSG (SEQ ID NO:52), GGNGSGSG (SEQ ID NO:53) and GGNGSG (SEQ ID NO:54). Peptide linkers of particular interest are (G4S) (SEQ ID NO:40), (G₄S)₂ or GGGGSGGGGS (SEQ ID NO:41), (G4S)₃ (SEQ ID NO:45) and (G4S)₄ (SEQ ID NO:46).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a heavy chain of an antibody and a Fab fragment) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the bispecific antigen binding molecules provided herein are contemplated. For example, it may be desirable to improve the binding affinity and/or other biological properties of the antigen binding molecules. Amino acid sequence variants of the bispecific antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the bispecific antigen binding molecules. Any combination of deletion, insertion, and substitution can be made to arrive at the final antigen binding molecule, provided that the final antigen binding molecule possesses the desired characteristics, e.g., antigen-binding. Sites of interest for substitutional mutagenesis include the CDRs and Framework (FRs). Conservative substitutions are provided in Table A under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| | | |
|---|---|---|
| Original Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more CDRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antigen binding molecules with an N-terminal methionyl residue. Other insertional variants of the molecule include the fusion to the N- or C-terminus to a polypeptide which increases the serum half-life of the antigen binding molecules.

In certain embodiments, the antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antigen binding molecules may be made in order to create variants with certain improved properties. In one aspect, variants of antigen binding molecules are provided having a carbohydrate structure that lacks fucose attached (directly or indirectly) to an Fc region. Such fucosylation variants may have improved ADCC function, see e.g. U.S. Pat. Publication Nos. US 2003/0157108 (Presta, L.) or US 2004/0093621 (Kyowa Hakko Kogyo Co., Ltd). Further variants of the antigen binding molecules of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the antigen binding molecules of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541. An “immunoconjugate” is an antibody conjugated to one or more heterologous molecule(s), including but not limited to a cytotoxic agent.

The term “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a bispecific antigen binding molecule or antibody” refers to one or more nucleic acid molecules encoding the heavy and light chains (or fragments thereof) of the bispecific antigen binding molecule or antibody, including such nucleic acid molecule(s) in a single vector or separate vectors, and such nucleic acid molecule(s) present at one or more locations in a host cell.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

The combination therapies in accordance with the invention have a synergistic effect. A “synergistic effect” of two compounds is one in which the effect of the combination of the two agents is greater than the sum of their individual effects and is statistically different from the controls and the single drugs. In another embodiment, the combination therapies disclosed herein have an additive effect. An “additive effect” of two compounds is one in which the effect of the combination of the two agents is the sum of their individual effects and is statistically different from either the controls and/or the single drugs.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The terms “cancer” and “cancerous” refer to or describe the physiological condition in mammals that is typically characterized by unregulated cell growth, i.e proliferative diseases, such as solid tumors or melanoma. A “tumor” comprises one or more cancerous cells. The term “solid tumor” as used herein refers to an abnormal mass of tissue that usually does not contain cysts or liquid areas. Solid tumors may be benign or malignant. Examples of cancer include, but are not limited to, carcinoma, lymphoma, blastoma, sarcoma, and leukemia or lymphoid malignancies. More particular examples of such cancers include breast cancer, squamous cell cancer (e.g., epithelial squamous cell cancer), lung cancer including small- cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. In a preferred embodiment, the cancer is head and neck cancer.

Reference to a tumor or cancer as a “Stage 0,” “Stage I,” “Stage II,” “Stage III,” or “Stage IV”, and various sub-stages within this classification, indicates classification of the tumor or cancer using the Overall Stage Grouping or Roman Numeral Staging methods known in the art. Although the actual stage of the cancer is dependent on the type of cancer, in general, a Stage 0 cancer is an in situ lesion, a Stage I cancer is small localized tumor, a Stage II and III cancer is a local advanced tumor which exhibits involvement of the local lymph nodes, and a Stage IV cancer represents metastatic cancer. The specific stages for each type of tumor is known to the skilled clinician.

An “advanced” cancer is one which has spread outside the site or organ of origin, either by local invasion or metastasis. Accordingly, the term “advanced” cancer includes both locally advanced and metastatic disease. A “refractory” cancer is one which progresses even though an anti-tumor agent, such as a chemotherapy, is being administered to the cancer patient. An example of a refractory cancer is one which is platinum refractory. A “recurrent” cancer is one which has regrown, either at the initial site or at a distant site, after a response to initial therapy, such as surgery. A “locally recurrent” cancer is cancer that returns after treatment in the same place as a previously treated cancer. An “operable” or “resectable” cancer is cancer which is confined to the primary organ and suitable for surgery (resection). A “non-resectable” or “unresectable” cancer is not able to be removed (resected) by surgery.

Preferably, a “patient” or “subject” is a human patient. The patient may be a “cancer patient,” i.e. one who is suffering or at risk for suffering from one or more symptoms of cancer, in particular head and neck cancer.

A “patient population” refers to a group of cancer patients. Such populations can be used to demonstrate statistically significant efficacy and/or safety of a drug, such as Pertuzumab. A “relapsed” patient is one who has signs or symptoms of cancer after remission. Optionally, the patient has relapsed after adjuvant or neoadjuvant therapy.

The terms “combination therapy” or “combined treatment” or “in combination” as used herein denote any form of concurrent or parallel treatment with at least two distinct therapeutic agents.

“Neoadjuvant therapy” or “preoperative therapy” herein refers to therapy given prior to surgery. The goal of neoadjuvant therapy is to provide immediate systemic treatment, potentially eradicating micrometastases that would otherwise proliferate if the standard sequence of surgery followed by systemic therapy were followed. Neoadjuvant therapy may also help to reduce tumor size thereby allowing complete resection of initially unresectable tumors or preserving portions of the organ and its functions. Furthermore, neoadjuvant therapy permits an in vivo assessment of drug efficacy, which may guide the choice of subsequent treatments.

“Adjuvant therapy” herein refers to therapy given after definitive surgery, where no evidence of residual disease can be detected, so as to reduce the risk of disease recurrence. The goal of adjuvant therapy is to prevent recurrence of the cancer, and therefore to reduce the chance of cancer-related death. Adjuvant therapy herein specifically excludes neoadjuvant therapy.

“Chemotherapy” refers to the use of a chemotherapeutic agent useful in the treatment of cancer.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors.

### Exemplary FAP-Targeted CD40 Agonists for Use in the Invention

Provided are bispecific agonistic CD40-antigen binding molecules comprising at least one antigen binding domain capable of specific binding to a tumor-associated antigen and their use in combination with radiotherapy, in particular their use in a method for treating a solid tumor in an individual.

In some aspects, the bispecific agonistic CD40-antigen binding molecule for use in treating a solid tumor in an individual is a bispecific agonistic CD40-antigen binding molecule comprising at least one antigen binding domain capable of specific binding to CD40 and at least one antigen binding domain capable of specific binding to Fibroblast Activation Protein (FAP), wherein the at least one antigen binding domain capable of specific binding to CD40 comprises a heavy chain variable region (V_{H}CD40) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}CD40) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6. In particular, the bispecific agonistic CD40-antigen binding molecule comprises at least one antigen binding domain capable of specific binding to CD40 comprising a VH comprising the amino acid sequence of SEQ ID NO:7 and a VL comprising the amino acid sequence of SEQ ID NO:8.

In further aspects, the bispecific agonistic CD40-antigen binding molecule comprises at least one antigen binding domain capable of specific binding to FAP comprising
(a) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or
(b) a heavy chain variable region (V_{H}FAP) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a light chain variable region (V_{L}FAP) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(c) a heavy chain variable region (V_{H}FAP) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}FAP) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In some aspects, the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:9, (ii) CDR-H2 comprising the amino acid sequence of SEQ ID NO: 10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14. In another aspects, the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a light chain variable region (V_{L}FAP) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22. In yet another aspects, the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}FAP) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

In some aspects, the bispecific agonistic CD40-antigen binding molecule comprises at least one antigen binding domain capable of specific binding to FAP comprising
(a) a heavy chain variable region (V_{H}FAP) comprising the amino acid sequence of SEQ ID NO:15 and a light chain variable region (V_{L}FAP) comprising the amino acid sequence of SEQ ID NO: 16,
(b) a heavy chain variable region (V_{H}FAP) comprising the amino acid sequence of SEQ ID NO:23 and a light chain variable region (V_{L}FAP) comprising the amino acid sequence of SEQ ID NO:24, or
(c) a heavy chain variable region (V_{H}FAP) comprising the amino acid sequence of SEQ ID NO:31 and a light chain variable region (V_{L}FAP) comprising the amino acid sequence of SEQ ID NO:32, or
(d) a heavy chain variable region (V_{H}FAP) comprising the amino acid sequence of SEQ ID NO:80 and a light chain variable region (V_{L}FAP) comprising the amino acid sequence of SEQ ID NO:81.

In one aspect, the bispecific agonistic CD40-antigen binding molecule comprises at least one antigen binding domain capable of specific binding to FAP comprising a heavy chain variable region (V_{H}FAP) comprising the amino acid sequence of SEQ ID NO:80 and a light chain variable region (V_{L}FAP) comprising the amino acid sequence of SEQ ID NO:81.

In particular aspects, the bispecific agonistic CD40-antigen binding molecule comprises at least one antigen binding domain capable of specific binding to FAP comprising a heavy chain variable region (V_{H}FAP) comprising the amino acid sequence of SEQ ID NO:15 and a light chain variable region (V_{L}FAP) comprising the amino acid sequence of SEQ ID NO:16.

Thus, in particular aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and (ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:16.

In other aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and (ii) at least one antigen binding domain capable of specific binding to FAP, comprising a heavy chain variable region (V_{H}FAP) comprising an amino acid sequence of SEQ ID NO:23 and a light chain variable region (V_{L}FAP) comprising an amino acid sequence of SEQ ID NO:24. In yet another aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:78 and a light chain variable region (V_{L}CD40) comprising the amino acid sequence of SEQ ID NO:79, and (ii) at least one antigen binding domain capable of specific binding to FAP, comprising a heavy chain variable region (V_{H}FAP) comprising an amino acid sequence of SEQ ID NO:23 and a light chain variable region (V_{L}FAP) comprising an amino acid sequence of SEQ ID NO:24.

In another aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and (ii) at least one antigen binding domain capable of specific binding to FAP, comprising a heavy chain variable region (V_{H}FAP) comprising an amino acid sequence of SEQ ID NO:23 and a light chain variable region (V_{L}FAP) comprising an amino acid sequence of SEQ ID NO:24. In other aspects, the bispecific agonistic CD40-antigen binding molecule comprises (i) at least one antigen binding domain capable of specific binding to CD40, comprising a heavy chain variable region (V_{H}CD40) comprising the amino acid sequence of SEQ ID NO: 7 and a light chain variable region (V_{L}CD40) comprising the amino acid sequence of SEQ ID NO: 8, and (ii) at least one antigen binding domain capable of specific binding to FAP, comprising a heavy chain variable region (V_{H}FAP) comprising an amino acid sequence of SEQ ID NO:31 and a light chain variable region (V_{L}FAP) comprising an amino acid sequence of SEQ ID NO:32.

In some aspects, the bispecific agonistic CD40-antigen binding molecule comprises
a) at least two Fab fragments capable of specific binding to CD40 fused at its C-terminus to the N-terminus of a Fc region, and
(b) one antigen binding domain capable of specific binding to FAP fused at its N-terminus to the C-terminus of the Fc region.

In some aspects, the bispecific agonistic CD40-antigen binding molecule comprises
a) at least two Fab fragments capable of specific binding to CD40 fused at its C-terminus to the N-terminus of a Fc region, and
(b) a cross-fab fragment capable of specific binding to FAP fused to the C-terminus of the Fc region.

In particular aspects, the bispecific agonistic CD40-antigen binding molecule comprises a cross-fab fragment capable of specific binding to FAP, wherein the VH-Ckappa chain of the cross-fab fragment is fused to the C-terminus of the Fc region. In particular aspects, the bispecific agonistic CD40-antigen binding molecule comprises two Fab fragments capable of specific binding to CD40 fused at its C-terminus to the N-terminus of a Fc region. In particular aspects, the bispecific agonistic CD40-antigen binding molecule is characterized by bivalent binding to CD40 and monovalent binding to FAP.

In one aspect, the bispecific agonistic CD40-antigen binding molecule comprises
(a) two light chains and two heavy chains of an antibody comprising two Fab fragments capable of specific binding to CD40 and the Fc domain, and
(b) a VH and VL domain capable of specific binding to a target cell antigen, wherein the VH domain and the VL domain are each connected via a peptide linker to one of the C-termini of the two heavy chains.

In a further aspect, provided is a bispecific agonistic CD40-antigen binding molecule, comprising
(a) two heavy chains, each heavy chain comprising a VH and CH1 domain of a Fab fragment capable of specific binding to CD40 and a Fc region subunit,
(b) two light chains, each light chain comprising a VL and CL domain of a Fab fragment capable of specific binding to CD40, and
(c) a cross-fab fragment capable of specific binding to FAP comprising a VL-CH1 chain and a VH-CL chain, wherein the VH-CL chain is connected to the C-terminus of one of the two heavy chains of (a).

In one aspect, the VH-CL (VH-Ckappa) chain is connected to the C-terminus of the Fc knob heavy chain.

In one particular aspect, the invention provides a bispecific antigen binding molecule comprising (a) two light chains, each comprising the amino acid sequence of SEQ ID NO:62, one light chain comprising the amino acid sequence of SEQ ID NO:61, a first heavy chain comprising the amino acid sequence of SEQ ID NO:63, and a second heavy chain comprising the amino acid sequence of SEQ ID NO:64.

In another aspects, the bispecific agonistic CD40-antigen binding molecule is characterized by tetravalent or trivalent binding to CD40 and monovalent binding to FAP. In some aspects, the bispecific agonistic CD40-antigen binding molecule is characterized by tetravalent binding to CD40 and monovalent binding to FAP. In some aspects, the bispecific agonistic CD40-antigen binding molecule comprises four Fab fragments capable of specific binding to CD40, wherein each two Fab fragments are fused to each other and fused at its C-terminus to the N-terminus of a Fc region.

### Radiotherapy

In aspects of the present invention, the FAP-targeted CD40 agonist described herein is used in combination with radiotherapy.

Radiotherapy or radiation therapy, often abbreviated RT, is a therapy using ionizing radiation, generally as part of cancer treatment to control or kill malignant cells and normally delivered by a linear accelerator. Radiotherapy may be curative in a number of types of cancer if they are localized to one area of the body. It may also be used as part of adjuvant therapy, to prevent tumor recurrence after surgery to remove a primary malignant tumor (for example, early stages of breast cancer). Radiation therapy is synergistic with chemotherapy, and has been used before, during, and after chemotherapy in susceptible cancers.

Radiotherapy is commonly applied to the cancerous tumor because of its ability to control cell growth. Ionizing radiation works by damaging the DNA of cancerous tissue leading to cellular death. To spare normal tissues (such as skin or organs which radiation must pass through to treat the tumor), shaped radiation beams are aimed from several angles of exposure to intersect at the tumor, providing a much larger absorbed dose there than in the surrounding healthy tissue. Besides the tumor itself, the radiation fields may also include the draining lymph nodes if they are clinically or radiologically involved with the tumor, or if there is thought to be a risk of subclinical malignant spread. It is necessary to include a margin of normal tissue around the tumor to allow for uncertainties in daily set-up and internal tumor motion. These uncertainties can be caused by internal movement and movement of external skin marks relative to the tumor position. The response of a cancer to radiation is described by its radiosensitivity. Highly radiosensitive cancer cells are rapidly killed by modest doses of radiation. These include leukemias, most lymphomas and germ cell tumors. The majority epithelial cancers are only moderately radiosensitive, and require a significantly higher dose of radiation (60-70 Gy) to achieve a radical cure. Some types of cancer are notably radioresistant, that is, much higher doses are required to produce a radical cure than may be safe in clinical practice. It is important to distinguish the radiosensitivity of a particular tumor, which to some extent is a laboratory measure, from the radiation “curability” of a cancer in actual clinical practice. For example, leukemias are not generally curable with radiation therapy, because they are disseminated through the body. Lymphoma may be radically curable if it is localised to one area of the body. Similarly, many of the common, moderately radioresponsive tumors are routinely treated with curative doses of radiation therapy if then, are at an early stage. Cancers such as skin cancer, head and neck cancer, breast cancer, non-small cell lung cancer, cervical cancer and prostate cancer are often incurable with radiotherapy because it is not possible to treat the whole body.

The response of a tumor to radiotherapy depends on the size of the tumor. Very large tumors respond less well to radiation than smaller tumors or microscopic disease. Various strategies are used to overcome this effect. The most common technique is surgical resection prior to radiation therapy. This is most commonly seen in the treatment of breast cancer with mastectomy followed by adjuvant radiotherapy. Another method is to shrink the tumor with neoadjuvant chemotherapy prior to radical radiation therapy. A third technique is to enhance the radiosensitivity of the cancer by giving certain drugs during a course of radiation therapy. Examples of radio sensitizing drugs include cisplatin, nimorazole and cetuximab.

Radiotherapy usually causes minimal or no side effects, although short-term pain flare-up can be experienced in the days following treatment due to oedema compressing nerves in the treated area. Higher doses can cause varying side effects during treatment (acute side effects), in the months or years following treatment (long-term side effects), or after re-treatment (cumulative side effects). The nature, severity, and longevity of side effects depends on the organs that receive the radiation, the treatment itself (type of radiation, dose, fractionation), and the patient. The main side effects reported are fatigue and skin irritation, like a mild to moderate sun burn. The fatigue often sets in during the middle of a course of treatment and can last for weeks after treatment ends. The irritated skin will heal, but may not be as elastic as it was before. Side effects from radiation are often limited to the area of the patient’s body that is under treatment and are dose-dependent. For example, higher doses of head and neck radiation can be associated with cardiovascular complications, thyroid dysfunction, and pituitary axis dysfunction. Thus, a lower dose of radiotherapy may be preferred.

The amount of radiation used in photon radiotherapy is measured in Grays (Gy), and varies depending on the type and stage of cancer being treated. For curative cases, the typical dose for a solid epithelial tumor ranges from 60 to 80 Gy, while lymphomas are treated with a range of 20 to 40 Gy. Preventive doses are typically in the range of 45 to 60 Gy in fractions of 1.8 to 2 Gy (for breast, head, and neck cancers.) Many other factors are considered by radiation oncologists when selecting a dose, including whether the patient is receiving chemotherapy, patient comorbidities, whether radiotherapy is being administered before or after surgery. Delivery parameters of a prescribed dose are determined during treatment planning (part of dosimetry). Treatment planning is generally performed on dedicated computers using specialized treatment planning software. Depending on the radiation delivery method, several angles or sources may be used to sum to the total necessary dose. The planner will try to design a plan that delivers a uniform prescription dose to the tumor and minimizes the dose to surrounding healthy tissues.

The total dose is fractionated (spread out over time) for several important reasons. Fractionation allows normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given. Similarly, tumor cells that were chronically or acutely hypoxic (and therefore more radioresistant) may reoxygenate between fractions, improving the tumor cell kill. The typical fractionation schedule for adults is 1.8 to 2 Gy per day, five days a week. In some cancer types, prolongation of the fraction schedule over too long can allow for the tumor to begin repopulating, and for these tumor types, including head-and-neck and cervical squamous cell cancers, radiation treatment is preferably completed within a certain amount of time. For children, a typical fraction size may be 1.5 to 1.8 Gy per day, as smaller fraction sizes are associated with reduced incidence and severity of late-onset side effects in normal tissues. In some cases, two fractions per day are used near the end of a course of treatment. This schedule, known as a concomitant boost regimen or hyperfractionation, is used on tumors that regenerate more quickly when they are smaller. In particular, tumors in the head-and-neck demonstrate this behavior.

One fractionation schedule that is increasingly being used and continues to be studied is hypofractionation. This is a radiation treatment in which the total dose of radiation is divided into large doses. Typical doses vary significantly by cancer type, from 1.8 Gy/fraction to 20 Gy/fraction, the latter being typical of stereotactic treatments (stereotactic ablative body radiotherapy, or SABR - also known as SBRT, or stereotactic body radiotherapy) for subcranial lesions, or SRS (stereotactic radiosurgery) for intracranial lesions. A hypofractioned radiation at a dose in a range of 5 to 20 Gy may be preferred. Depending on the cancer to be treated, a hypofractioned radiation at a dose in a range of 1.8 to 2.2 Gy may be of particular interest. The rationale of hypofractionation is to reduce the probability of local recurrence by denying clonogenic cells the time they require to reproduce and also to exploit the radiosensitivity of some tumors. In particular, stereotactic treatments are intended to destroy clonogenic cells by a process of ablation - i.e. the delivery of a dose intended to destroy clonogenic cells directly, rather than to interrupt the process of clonogenic cell division repeatedly (apoptosis), as in routine radiotherapy.

There are two forms of local radiotherapy, external beam radiotherapy and internal radiotherapy. External beam radiotherapy (EBRT) is the most common form of radiotherapy. An external source of ionizing radiation is pointed at a particular part of the body of the patient. In contrast to brachytherapy (sealed source radiotherapy) and unsealed source radiotherapy, in which the radiation source is inside the body, external beam radiotherapy directs the radiation at the tumor from outside the body. Orthovoltage (“superficial”) X-rays are used for treating skin cancer and superficial structures. X-rays and electron beams are by far the most widely used sources for external beam radiotherapy.

Internal radiotherapy (brachytherapy) is a form of radiotherapy where a sealed radiation source is placed inside or next to the area requiring treatment. The advantage of brachytherapy is that the irradiation affects only a very localized area around the radiation sources. Exposure to radiation of healthy tissues far away from the sources is therefore reduced and the tumor can be treated with very high doses of localised radiation whilst reducing the probability of unnecessary damage to surrounding healthy tissues. In addition, if the patient moves or if there is any movement of the tumor within the body during treatment, the radiation sources retain their correct position in relation to the tumor.

Systemic radiotherapy or radioisotope therapy (RIT) is another form of radiotherapy. Targeting can be achieved through the chemical properties of an isotope such as radioiodine which is specifically absorbed by the thyroid gland a thousandfold better than other organs in the body, or by attaching the radioisotope to another molecule or antibody to guide it to the target tissue. The radioisotopes are delivered through infusion (into the bloodstream) or ingestion. Examples are the infusion of metaiodobenzylguanidine (MIBG) to treat neuroblastoma, of oral iodine-131 to treat thyroid cancer or thyrotoxicosis, and of hormone-bound lutetium-177 and yttrium-90 to treat neuroendocrine tumors (peptide receptor radionuclide therapy). Another example is the injection of radioactive yttrium-90 or holmium-166 microspheres into the hepatic artery to radioembolize liver tumors or liver metastases. These microspheres are used for the treatment approach known as selective internal radiation therapy. The microspheres are approximately 30 µm in diameter (about one-third of a human hair) and are delivered directly into the artery supplying blood to the tumors. These treatments begin by guiding a catheter up through the femoral artery in the leg, navigating to the desired target site and administering treatment. The blood feeding the tumor will carry the microspheres directly to the tumor enabling a more selective approach than traditional systemic chemotherapy. There are currently three different kinds of microspheres: SIR-Spheres, TheraSphere and QuiremSpheres. A major use of systemic radioisotope therapy is in the treatment of bone metastasis from cancer. The radioisotopes travel selectively to areas of damaged bone, and spare normal undamaged bone. Isotopes commonly used in the treatment of bone metastasis are radium-223, strontium-89 and samarium (¹⁵³Sm) lexidronam.

### Preparation of Bispecific Antigen Binding Molecules for Use in the Invention

The therapeutic agents used in the combination comprise multispecific antigen binding molecules, e.g. bispecific antigen binding molecules. Multispecific antibodies are monoclonal antibodies that have binding specificities for at least two different sites. In certain aspects, the binding specificities are for different antigens. In certain aspects, the binding specificities are for different epitopes on the same antigen. Bispecific antigen binding molecules can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). For recombinant production one or more polynucleotide encoding the bispecific antigen binding molecule or polypeptide fragments thereof are provided. The one or more polynucleotide encoding the bispecific antigen binding molecule are isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the bispecific antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the bispecific antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and the 5′-flanking and internal regions of the rabbit alpha-globin gene, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions may be associated with additional coding regions which encode secretory or signal peptides, which direct the secretion of a polypeptide encoded by a polynucleotide. For example, if secretion of the bispecific antigen binding molecule or polypeptide fragments thereof is desired, DNA encoding a signal sequence may be placed upstream of the nucleic acid encoding the bispecific antigen binding molecule or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. DNA encoding a short protein sequence that could be used to facilitate later purification (e.g. a histidine tag) or assist in labeling the fusion protein may be included within or at the ends of the polynucleotide encoding a bispecific antigen binding molecule used in the invention or polypeptide fragments thereof. In one aspect, a host cell comprises (e.g. has been transformed or transfected with) a vector comprising a polynucleotide that encodes (part of) a bispecific antigen binding molecule used in the invention. As used herein, the term “host cell” refers to any kind of cellular system which can be engineered to generate bispecific antigen binding molecules or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr- CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain. A method of producing a bispecific antigen binding molecule used in the invention comprises culturing a host cell comprising polynucleotides encoding the bispecific antigen binding molecule or polypeptide fragments thereof, as provided herein, under conditions suitable for expression of the bispecific antigen binding molecule or polypeptide fragments thereof, and recovering the bispecific antigen binding molecule or polypeptide fragments thereof from the host cell (or host cell culture medium).

Bispecific antigen binding molecules used in the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the bispecific antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the examples. The purity of the bispecific antigen binding molecule or fragments thereof can be determined by any of a variety of well-known analytical methods including gel electrophoresis, high pressure liquid chromatography, and the like. For example, the bispecific antigen binding molecules expressed as described in the Examples were shown to be intact and properly assembled as demonstrated by reducing and non-reducing SDS-PAGE.

### FC Domain Modifications Reducing FC Receptor Binding and/or Effector Function

The Fc domain of the antigen binding molecules used in the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antigen binding molecules to cells expressing Fc receptors rather than to the preferred antigen-bearing cells. Accordingly, in particular aspects, the Fc domain of the antigen binding molecules of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcy receptor. In one aspect, the Fc receptor is a human Fc receptor. In a specific aspect, the Fc receptor is an activating human Fcy receptor, more specifically human FcγRIIIa, FcyRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of a bispecific antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. In a particular aspect, the invention provides a bispecific antigen binding molecule, wherein the Fc domain comprises one or more amino acid substitution that reduces binding to an Fc receptor, in particular towards Fcγ receptor.

In one aspect, the Fc domain of the bispecific antigen binding molecule comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is an antibody according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcy receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or cell activating antibodies comprising an Fc domain for Fc receptors may be evaluated using cell lines known to express particular Fc receptors, such as human NK cells expressing FcγIIIa receptor.

Effector function of an Fc domain, or bispecific antigen binding molecules comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some aspects, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention are able to bind C1q and hence have CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

### Fc Domain Modifications Promoting Heterodimerization

The bispecific antigen binding molecules used in the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antibodies of the invention in recombinant production, it will thus be advantageous to introduce in the Fc domain of the bispecific antigen binding molecules of the invention a modification promoting the association of the desired polypeptides. Accordingly, the bispecific antigen binding molecule comprises a Fc domain composed of a first and a second subunit capable of stable association, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the bispecific antigen binding molecule comprises a Fc domain composed of a first and a second subunit capable of stable association, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in US 5,731,168; US 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antigen binding molecule as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

### Modifications in the Fab Domains

In one aspect, the invention relates to a bispecific antigen binding molecule comprising (a) a first Fab fragment capable of specific binding to CD40, (b) a second Fab fragment capable of specific binding to FAP, and (c) a Fc domain composed of a first and a second subunit capable of stable association, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, the invention relates to a bispecific antigen binding molecule comprising (a) a first Fab fragment capable of specific binding to CD40, (b) a second Fab fragment capable of specific binding to FAP, and (c) a Fc domain composed of a first and a second subunit capable of stable association, wherein in one of the Fab fragments the constant domains CL and CH1 are replaced by each other so that the CH1 domain is part of the light chain and the CL domain is part of the heavy chain. More particularly, in the second Fab fragment capable of specific binding to a target cell antigen the constant domains CL and CH1 are replaced by each other so that the CH1 domain is part of the light chain and the CL domain is part of the heavy chain.

In a particular aspect, the invention relates a bispecific antigen binding molecule comprising (a) a first Fab fragment capable of specific binding to CD40, (b) a second Fab fragment capable of specific binding to FAP, wherein the constant domains CL and CH1 are replaced by each other so that the CH1 domain is part of the light chain and the CL domain is part of the heavy chain. Such a molecule binds monovalently to both CD40 and FAP.

In another aspect, the invention relates to a bispecific antigen binding molecule, comprising (a) two light chains and two heavy chains of an antibody comprising two Fab fragments capable of specific binding to CD40 and the Fc domain, and (b) two additional Fab fragments capable of specific binding to FAP, wherein said additional Fab fragments are each connected via a peptide linker to the C-terminus of the heavy chains of (a). In a particular aspect, the additional Fab fragments are Fab fragments, wherein the variable domains VL and VH are replaced by each other so that the VH domain is part of the light chain and the VL domain is part of the heavy chain.

Thus, in a particular aspect, the invention comprises a bispecific antigen binding molecule, comprising (a) two light chains and two heavy chains of an antibody comprising two Fab fragments capable of specific binding to CD40 and the Fc domain, and (b) two additional Fab fragments capable of specific binding to FAP, wherein said two additional Fab fragments capable of specific binding to a target cell antigen are crossover Fab fragments wherein the variable domains VL and VH are replaced by each other and the VL-CH chains are each connected via a peptide linker to the C-terminus of the heavy chains of (a).

In another aspect, and to further improve correct pairing, the bispecific antigen binding molecule comprising (a) a first Fab fragment capable of specific binding to CD40, (b) a second Fab fragment capable of specific binding to FAP, and (c) a Fc domain composed of a first and a second subunit capable of stable association, can contain different charged amino acid substitutions (so-called “charged residues”). These modifications are introduced in the crossed or non-crossed CH1 and CL domains. In a particular aspect, the invention relates to a bispecific antigen binding molecule, wherein in one of CL domains the amino acid at position 123 (EU numbering) has been replaced by arginine (R) and/or wherein the amino acid at position 124 (EU numbering) has been substituted by lysine (K) and wherein in one of the CH1 domains the amino acids at position 147 (EU numbering) and/or at position 213 (EU numbering) have been substituted by glutamic acid (E).

### Assays

The antigen binding molecules provided herein may be identified, screened for, or characterized for their physical/chemical properties and/or biological activities by various assays known in the art.

### 1. Binding Assay

Binding of the bispecific antigen binding molecule provided herein to the corresponding target expressing cells may be evaluated for example by using a murine fibroblast cell line expressing human Fibroblast Activation Protein (FAP) and flow cytometry (FACS) analysis. Binding of the bispecific antigen binding molecules provided herein to CD40 may be determined by using primary B cells.

### 2. Activity Assays

Bispecific antigen binding molecules of the invention are tested for biological activity. Biological activity may include efficacy and specificity of the bispecific antigen binding molecules. Efficacy and specificity are demonstrated by assays showing agonistic signaling through the CD40 receptor upon binding of the target antigen. Furthermore, in vitro T cell priming assays are conducted using dendritic cells (DCs) that have been incubated with the bispecific antigen binding molecules.

### Pharmaceutical Compositions, Formulations and Routes of Administation

In a further aspect, the invention provides pharmaceutical compositions comprising any of the bispecific antigen binding molecules provided herein, e.g., for use in any of the below therapeutic methods. In one embodiment, a pharmaceutical composition comprises any of the bispecific antigen binding molecules provided herein and at least one pharmaceutically acceptable excipient. In another embodiment, a pharmaceutical composition comprises any of the bispecific antigen binding molecules provided herein and at least one additional therapeutic agent, e.g., as described below.

Pharmaceutical compositions of the present invention comprise a therapeutically effective amount of one or more bispecific antigen binding molecules dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one bispecific antigen binding molecule according to the invention and optionally an additional active ingredient will be known to those of skill in the art in light of the present disclosure, as exemplified by Remington’s Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intra-lesional, intravenous, intra-arterial, intramuscular, intrathecal or intraperitoneal injection. For injection, the bispecific antigen binding molecules of the invention may be formulated in aqueous solutions, preferably in physiologically compatible buffers such as Hanks’ solution, Ringer’s solution, or physiological saline buffer. The solution may contain formulatory agents such as suspending, stabilizing and/or dispersing agents. Alternatively, the bispecific antigen binding molecules may be in powder form for constitution with a suitable vehicle, e.g., sterile pyrogen-free water, before use. Sterile injectable solutions are prepared by incorporating the antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the antigen binding molecules may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as emulsion in an acceptable oil) or ion exchange resins, or as sparingly soluble derivatives, for example, as a sparingly soluble salt.

Pharmaceutical compositions comprising the bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

### Administration of the FAP-Targeted CD40 Agonist in Combination With Radiotherapy

The bispecific agonistic CD40-antigen binding molecule comprising at least one antigen binding domain capable of specific binding to a tumor-associated antigen can be administered by any suitable means, including parenteral, intrapulmonary, and intranasal, and, if desired for local treatment, intralesional administration. In particular, the bispecific agonistic CD40-antigen binding molecule can be administered by parenteral, particularly intravenous, infusion.

Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In one aspect, the bispecific agonistic CD40-antigen binding molecule is administered parenterally, particularly intravenously. In a particular aspect, the bispecific agonistic CD40-antigen binding molecule is administerd by intravenous infusion.

The bispecific agonistic CD40-antigen binding molecule would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The bispecific agonistic CD40-antigen binding molecule need not be, but is optionally formulated with one or more agents currently used to prevent or treat the disorder in question. The effective amount of such other agents depends on the amount of therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the bispecific agonistic CD40-antigen binding molecule (when used in combination or with one or more other additional therapeutic agents) will depend on the type of disease to be treated, the severity and course of the disease, whether it is administered for preventive or therapeutic purposes, previous therapy, the patient’s clinical history and response to the therapeutic agent, and the discretion of the attending physician. The bispecific agonistic CD40-antigen binding molecule is suitably administered to the patient at one time or over a series of treatments. Depending on the type and severity of the disease, about 1 µg/kg to 15 mg/kg (e.g. 0.1 mg/kg - 10 mg/kg) of the substance can be an initial candidate dosage for administration to the subject, whether, for example, by one or more separate administrations, or by continuous infusion. One typical daily dosage might range from about 1 µg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific agonistic CD40-antigen binding molecule would be in the range from about 0.05 mg/kg to about 10 mg/kg. Thus, one or more doses of about 0.5 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg or 10 mg/kg (or any combination thereof) may be administered to the subject. Such doses may be administered intermittently, e.g. every week, every two weeks, or every three weeks (e.g. such that the subject receives from about two to about twenty, or e.g. about six doses of the therapeutic agent). An initial higher loading dose, followed by one or more lower doses, or an initial lower dose, followed by one or more higher doses may be administered. An exemplary dosing regimen comprises administering an initial dose of about 10 mg, followed by a bi-weekly dose of about 20 mg of the therapeutic agent.

However, other dosage regimens may be useful. The progress of this therapy is easily monitored by conventional techniques and assays.

In one aspect, the administration of the bispecific agonistic CD40-antigen binding molecule is a single administration. In some aspects, the bispecific agonistic CD40-antigen binding molecule is administered simultaneously with the radiotherapy. The term “simultaneously” means at the same time or within a short period of time, usually less than 1 hour. In some aspects, the bispecific agonistic CD40-antigen binding molecule is administered simultaneously after radiotherapy. “After radiotherapy” means within a period of more than 3 hours, preferably days after the radiation has finished, preferably after one day, however not more than 1 to 2 weeks after radiation has finished. In certain aspects, the administration of the the bispecific agonistic CD40-antigen binding molecule is two or more administrations. A drug that is administered “concurrently” with one or more other drugs is administered during the same treatment cycle, on the same day of treatment as the one or more other drugs, and, optionally, at the same time as the one or more other drugs. For instance, for therapies given every 3 weeks, the concurrently administered drugs are each administered on day-1 of a 3-week cycle. In one aspect, the administration of the the bispecific agonistic CD40-antigen binding molecule comprises an initial administration of a first dose of the the bispecific agonistic CD40-antigen binding molecule and one or more subsequent administrations of a second dose of the the bispecific agonistic CD40-antigen binding molecule wherein the first dose is not lower than the second dose.

When both radiotherapy and the bispecific agonistic CD40-antigen binding molecule are co-administered sequentially they are administered in two separate administrations that are separated by a “specific period of time”. The term specific period of time is meant anywhere from 1 hour to 15 days. For example, the bispecific agonistic CD40-antigen binding molecule can be administered within about 1, 2, 3, 4, 5, 6 or 7 days or 1 to 24 hours from the administration of radiotherapy, and, in one aspect, the specific period time is 1 to 3 days, or 2 to 8 hours.

In a particular aspect, radiotherapy is administered prior to the administration of the bispecific agonistic CD40-antigen binding molecule. In one aspect, radiotherapy is administered simultaneously to the administration of the bispecific agonistic CD40-antigen binding molecule.

### Therapeutic Methods and Compositions

In one aspect, provided is a method for treating a solid tumor in an individual comprising administering to the subject an effective amount of a bispecific agonistic CD40-antigen binding molecule described herein and an effective amount of radiotherapy decribed herein.

In one such aspect, the method further comprises administering to the indivividual an effective amount of at least one additional therapeutic agent. Suitable additional agents include one or more: anthracycline drugs, such as doxorubicin and epirubicin (pharmorubicin ®); taxane drugs, such as paclitaxel (Taxol®) and docetaxel (Taxotere®); fluorouracil (5FU); cyclophosphamide; methotrexate; cisplatin; and capecitabine. Other suitable additional therapeutic agents include the kinase inhibitors lapatinib (Tykerb) and neratinib (Nerlynx). In further embodiments, herein is provided a method for tumor shrinkage comprising administering to the subject an effective amount of a bispecific agonistic CD40-antigen binding molecule described herein and radiotherapy, as described herein. An “individual” or a “subject” according to any of the above aspects is preferably a human.

In one aspect, the invention provides a bispecific agonistic CD40-antigen binding molecule described herein for use in a method for treating or delaying progression of cancer, wherein the bispecific agonistic CD40-antigen binding molecule described herein is used in combination with radiotherapy. In some aspects, the bispecific agonistic CD40-antigen binding molecule described herein and the radiotherapy are combined with an additional therapeutic agent.

In further aspects, a combination therapy for use in cancer immunotherapy is provided comprising a bispecific agonistic CD40-antigen binding molecule described herein and radiotherapy as described herein.

In a further aspect, herein is provided is the use of a bispecific agonistic CD40-antigen binding molecule described herein in the manufacture or preparation of a medicament for use in combination with radiotherapy. In some aspects, the medicament is for treatment of cancer. In some embodiments, the medicament is for treatment of solid tumors. In some embodiments, the medicament is for use in a method of tumor shrinkage comprising administering to an individual having a solid tumor an effective amount of the medicament. In one such aspect, the method further comprises administering to the individual an effective amount of at least one additional therapeutic agent. In some aspects, the medicament is for treating solid tumors.

In particular aspects, the individual has cancer characterized by FAP-expression in the tumor stroma. In some aspects, the tumor may comprise a stroma overexpressing FAP. In some aspects, the cancer comprises a solid tumor. In some aspects, the solid tumor is an advanced and/or metastatic solid tumor. In some aspects, the FAP-expressing cancer is selected from the group consisting of head and neck cancer, melanoma, lung cancer, kidney cancer, breast cancer, colon cancer, ovarian cancer, cervical cancer, pancreatic cancer, liver cancer, prostate cancer, bladder cancer and gastric cancer. In some aspects, the cancer is head and neck cancer. In some aspects, the cancer is non-small cell lung cancer (NSCLC). In some aspects, the solid tumor is a tumor from a cancer relapsing after an earlier treatment. In some aspects, the tumor is from a head and neck cancer relapsing after an earlier treatment.

The treatment may be aimed at prevention of the development or growth of a solid tumor. As such, individuals may be prophylactically treated against development of a recurrence of the solid tumor using the bispecific agonistic CD40-antigen binding molecule described herein in combination with radiotherapy. In some aspects, the bispecific agonistic CD40-antigen binding molecule described herein acts synergistically with radiotherapy.

The bispecific agonistic CD40-antigen binding molecule described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### Articles of Manufacture (Kits)

In another aspect of the invention, a kit containing materials useful for the treatment, prevention and/or diagnosis of the disorders described above is provided. The kit comprises at least one container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). The active agent in the kit is the bispecific agonistic CD40-antigen binding molecule described herein. In addition, the kit includes instructions for using the bispecific agonistic CD40-antigen binding molecule described herein in combination radiotherapy. The label or package insert indicates how the composition is used for treating the condition of choice and provides the instructions for using the compositions in a combination therapy.

Alternatively, or additionally, the kit may further comprise a second (or third) container comprising a pharmaceutically-acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

TABLE B

(Sequences)

| SEQ ID NO: | Name | Sequence | | |
|---|---|---|---|---|
| 1 | hu CD40 CDR-H1 | GYYIH | | |
| 2 | hu CD40 CDR-H2 | RVIPNAGGTSYNQKFKG | | |
| 3 | hu CD40 CDR-H3 | EGIYW | | |
| 4 | hu CD40 CDR-L1 | RSSQSLVHSNGNTFLH | | |
| 5 | hu CD40 CDR-L2 | TVSNRFS | | |
| 6 | hu CD40 CDR-L3 | SQTTHVPWT | | |
| 7 | hu CD40 VH | QVQLVQSGAEVKKPGASVKVSCKASGYSFTGY YIHWVRQAPGQSLEWMGRVIPNAGGTSYNQKF KGRVTLTVDKSISTAYMELSRLRSDDTAVYYC AREGIYWWGQGTTVTVSS | | |
| 8 | hu CD40 VL | DIVMTQTPLSLSVTPGQPASISCRSSQSLVHS NGNTFLHWYLQKPGQSPQLLIYTVSNRFSGVP DRFSGSGSGTDFTLKISRVEAEDVGVYFCSQT THVPWTFGGGTKVEIK | | |
| 9 | FAP (212) CDR-H1 | DYNMD | | |
| 10 | FAP (212) CDR-H2 | DIYPNTGGTIYNQKFKG | | |
| 11 | FAP (212) CDR-H3 | FRGIHYAMDY | | |
| 12 | FAP (212) CDR-L1 | RASESVDNYGLSFIN | | |
| 13 | FAP (212) CDR-L2 | GTSNRGS | | |
| 14 | FAP (212) CDR-L3 | QQSNEVPYT | | |
| 15 | humanized FAP (212) VH | QVQLVQSGAEVKKPGASVKVSCKASGYTLTDY NMDWVRQAPGQGLEWIGDIYPNTGGTIYNQKF KGRVTMTIDTSTSTVYMELSSLRSEDTAVYYC TRFRGIHYAMDYWGQGTTVTVSS | | |
| 16 | humanized FAP (212) VL | EIVLTQSPATLSLSPGERATLSCRASESVDNY GLSFINWFQQKPGQAPRLLIYGTSNRGSGIPA RFSGSGSGTDFTLTISSLEPEDFAVYFCQQSN EVPYTFGGGTKVEIK | | |
| 17 | FAP (28H1) CDR-H1 | SHAMS | | |
| 18 | FAP (28H1) CDR-H2 | AIWASGEQYYADSVKG | | |
| 19 | FAP (28H1) CDR-H3 | GWLGNFDY | | |
| 20 | FAP (28H1) CDR-L1 | RASQSVSRSYLA | | |
| 21 | FAP (28H1) CDR-L2 | GASTRAT | | |
| 22 | FAP (28H1) CDR-L3 | QQGQVIPPT | | |
| 23 | FAP(28H1) VH | EVQLLESGGGLVQPGGSLRLSCAASGFTFSSHAMS WVRQAPGKGLEWVSAIWASGEQYYADSVKGRFTIS RDNSKNTLYLQMNSLRAEDTAVYYCAKGWLGNFDY WGQGTLVTVSS | | |
| 24 | FAP(28H1) VL | EIVLTQSPGTLSLSPGERATLSCRASQSVSRSYLA WYQQKPGQAPRLLIIGASTRATGIPDRFSGSGSGT DFTLTISRLEPEDFAVYYCQQGQVIPPTFGQGTKV EIK | | |
| 25 | FAP(4B9) CDR-H1 | SYAMS | | |
| 26 | FAP(4B9) CDR-H2 | AIIGSGASTYYADSVKG | | |
| 27 | FAP(4B9) CDR-H3 | GWFGGFNY | | |
| 28 | FAP(4B9) CDR-L1 | RASQSVTSSYLA | | |
| 29 | FAP(4B9) CDR-L2 | VGSRRAT | | |
| 30 | FAP(4B9) CDR-L3 | QQGIMLPPT | | |
| 31 | FAP(4B9) VH | EVQLLESGGGLVQPGGSLRLSCAASGFTFSSYAMS WVRQAPGKGLEWVSAIIGSGASTYYADSVKGRFTI SRDNSKNTLYLQMNSLRAEDTAVYYCAKGWFGGFN YWGQGTLVTVSS | | |
| 32 | FAP(4B9) VL | EIVLTQSPGTLSLSPGERATLSCRASQSVTSSYLA WYQQKPGQAPRLLINVGSRRATGIPDRFSGSGSGT DFTLTISRLEPEDFAVYYCQQGIMLPPTFGQGTKV EIK | | |
| 33 | hu CD40 | UniProt no. P25942, version 200 MVRLPLQCVL WGCLLTAVHP EPPTACREKQ YLINSQCCSL CQPGQKLVSD CTEFTETECL PCGESEFLDT WNRETHCHQH KYCDPNLGLR VQQKGTSETD TICTCEEGWH CTSEACESCV LHRSCSPGFG VKQIATGVSD TICEPCPVGF FSNVSSAFEK CHPWTSCETK DLVVQQAGTN KTDVVCGPQD RLRALVVIPI IFGILFAILL VLVFIKKVAK KPTNKAPHPK QEPQEINFPD DLPGSNTAAP VQETLHGCQP VTQEDGKESR ISVQERQ | | |
| 34 | hu FAP | UniProt no. Q12884, version 168 MKTWVKIVFG VATSAVLALL VMCIVLRPSR VHNSEENTMR ALTLKDILNG TFSYKTFFPN WISGQEYLHQ SADNNIVLYN IETGQSYTIL SNRTMKSVNA SNYGLSPDRQ FVYLESDYSK LWRYSYTATY YIYDLSNGEF VRGNELPRPI QYLCWSPVGS KLAYVYQNNI YLKQRPGDPP FQITFNGREN KIFNGIPDWV YEEEMLATKY ALWWSPNGKF LAYAEFNDTD IPVIAYSYYG DEQYPRTINI PYPKAGAKNP VVRIFIIDTT YPAYVGPQEV PVPAMIASSD YYFSWLTWVT DERVCLQWLK RVQNVSVLSI CDFREDWQTW DCPKTQEHIE ESRTGWAGGF FVSTPVFSYD AISYYKIFSD KDGYKHIHYI KDTVENAIQI TSGKWEAINI FRVTQDSLFY SSNEFEEYPG RRNIYRISIG SYPPSKKCVT CHLRKERCQY YTASFSDYAK YYALVCYGPG IPISTLHDGR TDQEIKILEE NKELENALKN IQLPKEEIKK LEVDEITLWY KMILPPQFDR SKKYPLLIQV YGGPCSQSVR SVFAVNWISY LASKEGMVIA LVDGRGTAFQ GDKLLYAVYR KLGVYEVEDQ ITAVRKFIEM GFIDEKRIAI WGWSYGGYVS SLALASGTGL FKCGIAVAPV SSWEYYASVY TERFMGLPTK DDNLEHYKNS TVMARAEYFR NVDYLLIHGT ADDNVHFQNS AQIAKALVNA | | |
| | | | QVDFQAMWYS DQNHGLSGLS TNHLYTHMTH FLKQCFSLSD | |
| 35 | hu FAP ectodomain+poly-lys-tag+his₆-tag | | RPSRVHNSEENTMRALTLKDILNGTFSYKTFFPNW ISGQEYLHQSADNNIVLYNIETGQSYTILSNRTMK SVNASNYGLSPDRQFVYLESDYSKLWRYSYTATYY IYDLSNGEFVRGNELPRPIQYLCWSPVGSKLAYVY QNNIYLKQRPGDPPFQITFNGRENKIFNGIPDWVY EEEMLATKYALWWSPNGKFLAYAEFNDTDIPVIAY SYYGDEQYPRTINIPYPKAGAKNPVVRIFIIDTTY PAYVGPQEVPVPAMIASSDYYFSWLTWVTDERVCL QWLKRVQNVSVLSICDFREDWQTWDCPKTQEHIEE SRTGWAGGFFVSTPVFSYDAISYYKIFSDKDGYKH IHYIKDTVENAIQITSGKWEAINIFRVTQDSLFYS SNEFEEYPGRRNIYRISIGSYPPSKKCVTCHLRKE RCQYYTASFSDYAKYYALVCYGPGIPISTLHDGRT DQEIKILEENKELENALKNIQLPKEEIKKLEVDEI TLWYKMILPPQFDRSKKYPLLIQVYGGPCSQSVRS VFAVNWISYLASKEGMVIALVDGRGTAFQGDKLLY AVYRKLGVYEVEDQITAVRKFIEMGFIDEKRIAIW GWSYGGYVSSLALASGTGLFKCGIAVAPVSSWEYY ASVYTERFMGLPTKDDNLEHYKNSTVMARAEYFRN VDYLLIHGTADDNVHFQNSAQIAKALVNAQVDFQA MWYSDQNHGLSGLSTNHLYTHMTHFLKQCFSLSDG KKKKKKGHHHHHH | |
| 36 | mouse FAP | | UniProt no. P97321 | |
| 37 | Murine FAP ectodomain+poly-lys-tag+his₆-tag | | RPSRVYKPEGNTKRALTLKDILNGTFSYKTYFPNW ISEQEYLHQSEDDNIVFYNIETRESYIILSNSTMK SVNATDYGLSPDRQFVYLESDYSKLWRYSYTATYY IYDLQNGEFVRGYELPRPIQYLCWSPVGSKLAYVY QNNIYLKQRPGDPPFQITYTGRENRIFNGIPDWVY EEEMLATKYALWWSPDGKFLAYVEFNDSDIPIIAY SYYGDGQYPRTINIPYPKAGAKNPVVRVFIVDTTY PHHVGPMEVPVPEMIASSDYYFSWLTWVSSERVCL QWLKRVQNVSVLSICDFREDWHAWECPKNQEHVEE SRTGWAGGFFVSTPAFSQDATSYYKIFSDKDGYKH IHYIKDTVENAIQITSGKWEAIYIFRVTQDSLFYS SNEFEGYPGRRNIYRISIGNSPPSKKCVTCHLRKE RCQYYTASFSYKAKYYALVCYGPGLPISTLHDGRT DQEIQVLEENKELENSLRNIQLPKVEIKKLKDGGL TFWYKMILPPQFDRSKKYPLLIQVYGGPCSQSVKS VFAVNWITYLASKEGIVIALVDGRGTAFQGDKFLH AVYRKLGVYEVEDQLTAVRKFIEMGFIDEERIAIW GWSYGGYVSSLALASGTGLFKCGIAVAPVSSWEYY ASIYSERFMGLPTKDDNLEHYKNSTVMARAEYFRN VDYLLIHGTADDNVHFQNSAQIAKALVNAQVDFQA MWYSDQNHGILSGRSQNHLYTHMTHFLKQCFSLSD GKKKKKKGHHHHHH | |
| 38 | Cynomolgus FAP ectodomain+poly-lys-tag+his₆-tag | RPPRVHNSEENTMRALTLKDILNGTFSYKTFFPNW ISGQEYLHQSADNNIVLYNIETGQSYTILSNRTMK SVNASNYGLSPDRQFVYLESDYSKLWRYSYTATYY IYDLSNGEFVRGNELPRPIQYLCWSPVGSKLAYVY QNNIYLKQRPGDPPFQITFNGRENKIFNGIPDWVY EEEMLATKYALWWSPNGKFLAYAEFNDTDIPVIAY SYYGDEQYPRTINIPYPKAGAKNPFVRIFIIDTTY PAYVGPQEVPVPAMIASSDYYFSWLTWVTDERVCL QWLKRVQNVSVLSICDFREDWQTWDCPKTQEHIEE SRTGWAGGFFVSTPVFSYDAISYYKIFSDKDGYKH IHYIKDTVENAIQITSGKWEAINIFRVTQDSLFYS SNEFEDYPGRRNIYRISIGSYPPSKKCVTCHLRKE RCQYYTASFSDYAKYYALVCYGPGIPISTLHDGRT DQEIKILEENKELENALKNIQLPKEEIKKLEVDEI TLWYKMILPPQFDRSKKYPLLIQVYGGPCSQSVRS VFAVNWISYLASKEGMVIALVDGRGTAFQGDKLLY AVYRKLGVYEVEDQITAVRKFIEMGFIDEKRIAIW GWSYGGYVSSLALASGTGLFKCGIAVAPVSSWEYY ASVYTERFMGLPTKDDNLEHYKNSTVMARAEYFRN VDYLLIHGTADDNVHFQNSAQIAKALVNAQVDFQA MWYSDQNHGLSGLSTNHLYTHMTHFLKQCFSLSDG KKKKKKGHHHHHH | | |
| 39 | murine CD40 | UniProt P27512, version 160 MVSLPRLCAL WGCLLTAVHL GQCVTCSDKQ YLHDGQCCDL CQPGSRLTSH CTALEKTQCH PCDSGEFSAQ WNREIRCHQH RHCEPNQGLR VKKEGTAESD TVCTCKEGQH CTSKDCEACA QHTPCIPGFG VMEMATETTD TVCHPCPVGF FSNQSSLFEK CYPWTSCEDK NLEVLQKGTS QTNVICGLKS RMRALLVIPV VMGILITIFG VFLYIKKVVK KPKDNEILPP AARRQDPQEM EDYPGHNTAA PVQETLHGCQ PVTQEDGKES RISVQERQVT DSIALRPLV | | |
| 40 | Peptide linker (G4S) | GGGGS | | |
| 41 | Peptide linker (G4S)₂ | GGGGSGGGGS | | |
| 42 | Peptide linker (SG4)₂ | SGGGGSGGGG | | |
| 43 | Peptide linker G4(SG4)₂ | GGGGSGGGGSGGGG | | |
| 44 | peptide linker | GSPGSSSSGS | | |
| 45 | (G4S)₃ peptide linker | GGGGSGGGGSGGGGS₃ | | |
| 46 | (G4S)₄ peptide linker | GGGGSGGGGSGGGGSGGGGS | | |
| 47 | peptide linker | GSGSGSGS | | |
| 48 | peptide linker | GSGSGNGS | | |
| 49 | peptide linker | GGSGSGSG | | |
| 50 | peptide linker | GGSGSG | | |
| 51 | peptide linker | GGSG | | |
| 52 | peptide linker | GGSGNGSG | | |
| 53 | peptide linker | | GGNGSGSG | |
| 54 | peptide linker | | GGNGSG | |
| 55 | mu CD40 CDR-H1 | | DYYMA | |
| 56 | mu CD40 CDR-H2 | | SISYDGSSTYYRDSVKG | |
| 57 | mu CD40 CDR-H3 | | HSSYFDY | |
| 58 | mu CD40 CDR-L1 | | RASDSVSTLMH | |
| 59 | mu CD40 CDR-L2 | | LASHLES | |
| 60 | mu CD40 CDR-L3 | | QQSWNDPWT | |
| 61 | (P1AE1689) light chain cross VH-Ckappa | | QVQLVQSGAEVKKPGASVKVSCKASGYTLTDYNMD WVRQAPGQGLEWIGDIYPNTGGTIYNQKFKGRVTM TIDTSTSTVYMELSSLRSEDTAVYYCTRFRGIHYA MDYWGQGTTVTVSSASVAAPSVFIFPPSDEQLKSG TASVVCLLNNFYPREAKVQWKVDNALQSGNSQESV TEQDSKDSTYSLSSTLTLSKADYEKHKVYACEVTH QGLSSPVTKSFNRGEC | |
| 62 | VL (CD40) light chain (charged) | | DIVMTQTPLSLSVTPGQPASISCRSSQSLVHSNGN TFLHWYLQKPGQSPQLLIYTVSNRFSGVPDRFSGS GSGTDFTLKISRVEAEDVGVYFCSQTTHVPWTFGG GTKVEIKRTVAAPSVFIFPPSDRKLKSGTASVVCL LNNFYPREAKVQWKVDNALQSGNSQESVTEQDSKD STYSLSSTLTLSKADYEKHKVYACEVTHQGLSSPV TKSFNRGEC | |
| 63 | VH (CD40) (VHCH1 charged) Fc knob_PGLALA_(P1AE168 9) (VL-CH1) | | QVQLVQSGAEVKKPGASVKVSCKASGYSFTGYYIH WVRQAPGQSLEWMGRVIPNAGGTSYNQKFKGRVTL TVDKSISTAYMELSRLRSDDTAVYYCAREGIYWWG QGTTVTVSSASTKGPSVFPLAPSSKSTSGGTAALG CLVEDYFPEPVTVSWNSGALTSGVHTFPAVLQSSG LYSLSSVVTVPSSSLGTQTYICNVNHKPSNTKVDE KVEPKSCDKTHTCPPCPAPEAAGGPSVFLFPPKPK DTLMISRTPEVTCVVVDVSHEDPEVKFNWYVDGVE VHNAKTKPREEQYNSTYRVVSVLTVLHQDWLNGKE YKCKVSNKALGAPIEKTISKAKGQPREPQVYTLPP CRDELTKNQVSLWCLVKGFYPSDIAVEWESNGQPE NNYKTTPPVLDSDGSFFLYSKLTVDKSRWQQGNVF SCSVMHEALHNHYTQKSLSLSPGGGGGSGGGGSGG GGSGGGGSEIVLTQSPATLSLSPGERATLSCRASE SVDNYGLSFINWFQQKPGQAPRLLIYGTSNRGSGI PARFSGSGSGTDFTLTISSLEPEDFAVYFCQQSNE VPYTFGGGTKVEIKSSASTKGPSVFPLAPSSKSTS GGTAALGCLVKDYFPEPVTVSWNSGALTSGVHTFP AVLQSSGLYSLSSVVTVPSSSLGTQTYICNVNHKP SNTKVDKKVEPKSC | |
| 64 | VH (CD40) (VHCH1 charged) Fc hole_PGLALA | | QVQLVQSGAEVKKPGASVKVSCKASGYSFTGYYIH WVRQAPGQSLEWMGRVIPNAGGTSYNQKFKGRVTL TVDKSISTAYMELSRLRSDDTAVYYCAREGIYWWG QGTTVTVSSASTKGPSVFPLAPSSKSTSGGTAALG CLVEDYFPEPVTVSWNSGALTSGVHTFPAVLQSSG | |
| | | | | LYSLSSVVTVPSSSLGTQTYICNVNHKPSNTKVDE KVEPKSCDKTHTCPPCPAPEAAGGPSVFLFPPKPK DTLMISRTPEVTCVVVDVSHEDPEVKFNWYVDGVE VHNAKTKPREEQYNSTYRVVSVLTVLHQDWLNGKE YKCKVSNKALGAPIEKTISKAKGQPREPQVCTLPP SRDELTKNQVSLSCAVKGFYPSDIAVEWESNGQPE NNYKTTPPVLDSDGSFFLVSKLTVDKSRWQQGNVF SCSVMHEALHNHYTQKSLSLSPG |
| 65 | | VH (CD40) (VHCH1 charged_VH1a (CD40) (VHCH1 charged)-Fc knob_PGLALA_(P1AE168 9) (VL-CH1) | | QVQLVQSGAEVKKPGASVKVSCKASGYSFTGYYIH WVRQAPGQSLEWMGRVIPNAGGTSYNQKFKGRVTL TVDKSISTAYMELSRLRSDDTAVYYCAREGIYWWG QGTTVTVSSASTKGPSVFPLAPSSKSTSGGTAALG CLVEDYFPEPVTVSWNSGALTSGVHTFPAVLQSSG LYSLSSVVTVPSSSLGTQTYICNVNHKPSNTKVDE KVEPKSCDGGGGSGGGGSQVQLVQSGAEVKKPGAS VKVSCKASGYSFTGYYIHWVRQAPGQSLEWMGRVI PNAGGTSYNQKFKGRVTLTVDKSISTAYMELSRLR SDDTAVYYCAREGIYWWGQGTTVTVSSASTKGPSV FPLAPSSKSTSGGTAALGCLVEDYFPEPVTVSWNS GALTSGVHTFPAVLQSSGLYSLSSVVTVPSSSLGT QTYICNVNHKPSNTKVDEKVEPKSCDKTHTCPPCP APEAAGGPSVFLFPPKPKDTLMISRTPEVTCVVVD VSHEDPEVKFNWYVDGVEVHNAKTKPREEQYNSTY RVVSVLTVLHQDWLNGKEYKCKVSNKALGAPIEKT ISKAKGQPREPQVYTLPPCRDELTKNQVSLWCLVK GFYPSDIAVEWESNGQPENNYKTTPPVLDSDGSFF LYSKLTVDKSRWQQGNVFSCSVMHEALHNHYTQKS LSLSPGGGGGSGGGGSGGGGSGGGGSEIVLTQSPA TLSLSPGERATLSCRASESVDNYGLSFINWFQQKP GQAPRLLIYGTSNRGSGIPARFSGSGSGTDFTLTI SSLEPEDFAVYFCQQSNEVPYTFGGGTKVEIKSSA STKGPSVFPLAPSSKSTSGGTAALGCLVKDYFPEP VTVSWNSGALTSGVHTFPAVLQSSGLYSLSSVVTV PSSSLGTQTYICNVNHKPSNTKVDKKVEPKSC |
| 66 | | VH (CD40) (VHCH1 charged)_VH (CD40) (VHCH1 charged)-Fc hole_PGLALA | | QVQLVQSGAEVKKPGASVKVSCKASGYSFTGYYIH WVRQAPGQSLEWMGRVIPNAGGTSYNQKFKGRVTL TVDKSISTAYMELSRLRSDDTAVYYCAREGIYWWG QGTTVTVSSASTKGPSVFPLAPSSKSTSGGTAALG CLVEDYFPEPVTVSWNSGALTSGVHTFPAVLQSSG LYSLSSVVTVPSSSLGTQTYICNVNHKPSNTKVDE KVEPKSCDGGGGSGGGGSQVQLVQSGAEVKKPGAS VKVSCKASGYSFTGYYIHWVRQAPGQSLEWMGRVI PNAGGTSYNQKFKGRVTLTVDKSISTAYMELSRLR SDDTAVYYCAREGIYWWGQGTTVTVSSASTKGPSV FPLAPSSKSTSGGTAALGCLVEDYFPEPVTVSWNS GALTSGVHTFPAVLQSSGLYSLSSVVTVPSSSLGT QTYICNVNHKPSNTKVDEKVEPKSCDKTHTCPPCP APEAAGGPSVFLFPPKPKDTLMISRTPEVTCVVVD VSHEDPEVKFNWYVDGVEVHNAKTKPREEQYNSTY |
| | | RVVSVLTVLHQDWLNGKEYKCKVSNKALGAPIEKT ISKAKGQPREPQVCTLPPSRDELTKNQVSLSCAVK GFYPSDIAVEWESNGQPENNYKTTPPVLDSDGSFF LVSKLTVDKSRWQQGNVFSCSVMHEALHNHYTQKS LSLSPG | | |
| 67 | 28H1 light chain cross VH-Ckappa | EVQLLESGGGLVQPGGSLRLSCAASGFTFSSHAMS WVRQAPGKGLEWVSAIWASGEQYYADSVKGRFTIS RDNSKNTLYLQMNSLRAEDTAVYYCAKGWLGNFDY WGQGTLVTVSSASVAAPSVFIFPPSDEQLKSGTAS VVCLLNNFYPREAKVQWKVDNALQSGNSQESVTEQ DSKDSTYSLSSTLTLSKADYEKHKVYACEVTHQGL SSPVTKSFNRGEC | | |
| 68 | VH (CD40) (VHCH1 charged) Fc knob_PGLALA_28H1 (VL-CH1) | QVQLVQSGAEVKKPGASVKVSCKASGYSFTGYYIH WVRQAPGQSLEWMGRVIPNAGGTSYNQKFKGRVTL TVDKSISTAYMELSRLRSDDTAVYYCAREGIYWWG QGTTVTVSSASTKGPSVFPLAPSSKSTSGGTAALG CLVEDYFPEPVTVSWNSGALTSGVHTFPAVLQSSG LYSLSSVVTVPSSSLGTQTYICNVNHKPSNTKVDE KVEPKSCDKTHTCPPCPAPEAAGGPSVFLFPPKPK DTLMISRTPEVTCVVVDVSHEDPEVKFNWYVDGVE VHNAKTKPREEQYNSTYRVVSVLTVLHQDWLNGKE YKCKVSNKALGAPIEKTISKAKGQPREPQVYTLPP CRDELTKNQVSLWCLVKGFYPSDIAVEWESNGQPE NNYKTTPPVLDSDGSFFLYSKLTVDKSRWQQGNVF SCSVMHEALHNHYTQKSLSLSPGGGGGSGGGGSGG GGSGGGGSEIVLTQSPGTLSLSPGERATLSCRASQ SVSRSYLAWYQQKPGQAPRLLIIGASTRATGIPDR FSGSGSGTDFTLTISRLEPEDFAVYYCQQGQVIPP TFGQGTKVEIKSSASTKGPSVFPLAPSSKSTSGGT AALGCLVKDYFPEPVTVSWNSGALTSGVHTFPAVL QSSGLYSLSSVVTVPSSSLGTQTYICNVNHKPSNT KVDKKVEPKSC | | |
| 69 | muCD40 (FGK4.5) light chain | DTVLTQSPAL AVSPGERVTI SCRASDSVST LMHWYQQKPG QQPKLLIYLA SHLESGVPAR FSGSGSGTDF TLTIDPVEAD DTATYYCQQS WNDPWTFGGG TKLELKRTVA APSVFIFPPS DEQLKSGTAS VVCLLNNFYP REAKVQWKVD NALQSGNSQE SVTEQDSKDS TYSLSSTLTL SKADYEKHKV YACEVTHQGL SSPVTKSFNR GEC | | |
| 70 | muCD40 VHCH1 charged_muCD40 VHCH1 charged-Fc knob_PGLALA_VH (28H1) | EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD GGGGSGGGGS EVQLVESDGG LVQPGRSLKL PCAASGFTFS DYYMAWVRQA PTKGLEWVAS ISYDGSSTYY RDSVKGRFTI | | |
| | SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHTCPPCPA PEAAGGPSVF LFPPKPKDTL MISRTPEVTC VVVDVSHEDP EVKFNWYVDG VEVHNAKTKP REEQYNSTYR VVSVLTVLHQ DWLNGKEYKC KVSNKALGAP IEKTISKAKG QPREPQVYTL PPCRDELTKN QVSLWCLVKG FYPSDIAVEW ESNGQPENNY KTTPPVLDSD GSFFLYSKLT VDKSRWQQGN VFSCSVMHEA LHNHYTQKSL SLSPGGGGGS GGGGSGGGGS GGGGSEVQLL ESGGGLVQPG GSLRLSCAAS GFTFSSHAMS WVRQAPGKGL EWVSAIWASG EQYYADSVKG RFTISRDNSK NTLYLQMNSL RAEDTAVYYC AKGWLGNFDY WGQGTLVTVS S | | | |
| 71 | EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD GGGGSGGGGS EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHTCPPCPA PEAAGGPSVF LFPPKPKDTL MISRTPEVTC VVVDVSHEDP EVKFNWYVDG VEVHNAKTKP REEQYNSTYR VVSVLTVLHQ DWLNGKEYKC KVSNKALGAP IEKTISKAKG QPREPQVCTL PPSRDELTKN QVSLSCAVKG FYPSDIAVEW ESNGQPENNY KTTPPVLDSD GSFFLVSKLT VDKSRWQQGN VFSCSVMHEA LHNHYTQKSL SLSPGGGGGS GGGGSGGGGS GGGGSEIVLT QSPGTLSLSP GERATLSCRA SQSVSRSYLA WYQQKPGQAP RLLIIGASTR ATGIPDRFSG SGSGTDFTLT ISRLEPEDFA VYYCQQGQVI PPTFGQGTVK EIK | | | |
| 72 | EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP | | | |
| | | EPVTVSWNSG ALTSGVHTFP AVLQSSGLYS LSSVVTVPSS SLGTQTYICN VNHKPSNTKV DKKVEPKSCD GGGGSGGGGS EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHTCPPCPA PEAAGGPSVF LFPPKPKDTL MISRTPEVTC VVVDVSHEDP EVKFNWYVDG VEVHNAKTKP REEQYNSTYR VVSVLTVLHQ DWLNGKEYKC KVSNKALGAP IEKTISKAKG QPREPQVYTL PPCRDELTKN QVSLWCLVKG FYPSDIAVEW ESNGQPENNY KTTPPVLDSD GSFFLYSKLT VDKSRWQQGN VFSCSVMHEA LHNHYTQKSL SLSPGGGGGS GGGGSGGGGS GGGGSEVQLL ESGGGLVQPG GSLRLSCAAS GFTFSSYAMS WVRQAPGKGL EWVSAISGSG GSTYYADSVK GRFTISRDNS KNTLYLQMNS LRAEDTAVYY CAKGSGFDYW GQGTLVTVSS | | |
| 73 | muCD40 VHCH1 charged_muCD40 VHCH1 charged-Fc knob_PGLALA_VL (DP47) | EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD GGGGSGGGGS EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSSASTK GPSVFPLAPS SKSTSGGTAA LGCLVKDYFP EPVTVSWNSG ALTSGVHTFP AVLQSSGLYS LSSVVTVPSS SLGTQTYICN VNHKPSNTKV DKKVEPKSCD KTHTCPPCPA PEAAGGPSVF LFPPKPKDTL MISRTPEVTC VVVDVSHEDP EVKFNWYVDG VEVHNAKTKP REEQYNSTYR VVSVLTVLHQ DWLNGKEYKC KVSNKALGAP IEKTISKAKG QPREPQVCTL PPSRDELTKN QVSLSCAVKG FYPSDIAVEW ESNGQPENNY KTTPPVLDSD GSFFLVSKLT VDKSRWQQGN VFSCSVMHEA LHNHYTQKSL SLSPGGGGGS GGGGSGGGGS GGGGSEIVLT QSPGTLSLSP GERATLSCRA SQSVSSSYLA WYQQKPGQAP RLLIYGASSR ATGIPDRFSG SGSGTDFTLT ISRLEPEDFA VYYCQQYGSS PLTFGQGTKV EIK | | |
| 74 | EVQLLESGGG LVQPGGSLRL SCAASGFTFS SHAMSWVRQA PGKGLEWVSA IWASGEQYYA DSVKGRFTIS RDNSKNTLYL QMNSLRAEDT AVYYCAKGWL GNFDYWGQGT LVTVSSASDA APTVSIFPPS SEQLTSGGAS VVCFLNNFYP KDINVKWKID GSERQNGVLN SWTDQDSKDS TYSMSSTLTL TKDEYERHNS YTCEATHKTS TSPIVKSFNR NEC | | | |
| 75 | DIVMTQTPLS LSVTPGQPAS ISCRSSQSLV HSNGNTFLHW YLQKPGQSPQ LLIYTVSNRF SGVPDRFSGS GSGTDFTLKI SRVEAEDVGV YFCSQTTHVP WTFGGGTKVE IKRADAAPTV SIFPPSSRKL TSGGASVVCF LNNFYPKDIN VKWKIDGSER QNGVLNSWTD QDSKDSTYSM SSTLTLTKDE YERHNSYTCE ATHKTSTSPI VKSFNRNEC | | | |
| 76 | QVQLVQSGAE VKKPGASVKV SCKASGYSFT GYYIHWVRQA PGQSLEWMGR VIPNAGGTSY NQKFKGRVTL TVDKSISTAY MELSRLRSDD TAVYYCAREG IYWWGQGTTV TVSSAKTTPP SVYPLAPGSA AQTNSMVTLG CLVEGYFPEP VTVTWNSGSL SSGVHTFPAV LQSDLYTLSS SVTVPSSTWP SQTVTCNVAH PASSTKVDEK IVPRDCGCKP CICTVPEVSS VFIFPPKPKD VLTITLTPKV TCVVVAISKD DPEVQFSWFV DDVEVHTAQT KPREEQINST FRSVSELPIM HQDWLNGKEF KCRVNSAAFG APIEKTISKT KGRPKAPQVY TIPPPKEQMA KDKVSLTCMI TNFFPEDITV EWQWNGQPAE NYDNTQPIMD TDGSYFVYSD LNVQKSNWEA GNTFTCSVLH EGLHNHHTEK SLSHSPGGGG GSGGGGSGGG GSGGGGSEIV LTQSPGTLSL SPGERATLSC RASQSVSRSY LAWYQQKPGQ APRLLIIGAS TRATGIPDRF SGSGSGTDFT LTISRLEPED FAVYYCQQGQ VIPPTFGQGT KVEIKSSAKT TPPSVYPLAP GSAAQTNSMV TLGCLVKGYF PEPVTVTWNS GSLSSGVHTF PAVLQSDLYT LSSSVTVPSS TWPSQTVTCN VAHPASSTKV DKKIVPRDC | | | |
| 77 | QVQLVQSGAE VKKPGASVKV SCKASGYSFT GYYIHWVRQA PGQSLEWMGR VIPNAGGTSY NQKFKGRVTL TVDKSISTAY MELSRLRSDD TAVYYCAREG IYWWGQGTTV TVSSAKTTPP SVYPLAPGSA AQTNSMVTLG CLVEGYFPEP VTVTWNSGSL SSGVHTFPAV LQSDLYTLSS SVTVPSSTWP SQTVTCNVAH PASSTKVDEK IVPRDCGCKP CICTVPEVSS VFIFPPKPKD VLTITLTPKV TCVVVAISKD DPEVQFSWFV | | | |
| | | DDVEVHTAQT KPREEQINST FRSVSELPIM HQDWLNGKEF KCRVNSAAFG APIEKTISKT KGRPKAPQVY TIPPPKKQMA KDKVSLTCMI TNFFPEDITV EWQWNGQPAE NYKNTQPIMK TDGSYFVYSK LNVQKSNWEA GNTFTCSVLH EGLHNHHTEK SLSHSPG | | |
| 78 | mu CD40 VH | EVQLVESDGG LVQPGRSLKL PCAASGFTFS DYYMAWVRQA PTKGLEWVAS ISYDGSSTYY RDSVKGRFTI SRDNAKSTLY LQMDSLRSED TATYYCGRHS SYFDYWGQGV MVTVSS | | |
| 79 | mu CD40 VL | DTVLTQSPAL AVSPGERVTI SCRASDSVST LMHWYQQKPG QQPKLLIYLA SHLESGVPAR FSGSGSGTDF TLTIDPVEAD DTATYYCQQS WNDPWTFGGG TKLELK | | |
| 80 | FAP (212) VH | EVLLQQSGPELVKPGASVKIACKASGYTLTDY NMDWVRQSHGKSLEWIGDIYPNTGGTIYNQKF KGKATLTIDKSSSTAYMDLRSLTSEDTAVYYC TRFRGIHYAMDYWGQGTSVTVSS | | |
| 81 | FAP (212) VL | DIVLTQSPVSLAVSLGQRATISCRASESVDNY GLSFINWFQQKPGQPPKLLIYGTSNRGSGVPA RFSGSGSGTDFSLNIHPMEEDDTAMYFCQQSN EVPYTFGGGTNLEIK | | |

General information regarding the nucleotide sequences of human immunoglobulins light and heavy chains is given in: Kabat, E.A., et al., Sequences of Proteins of Immunological Interest, 5th ed., Public Health Service, National Institutes of Health, Bethesda, MD (1991). Amino acids of antibody chains are numbered and referred to according to the numbering systems according to Kabat (Kabat, E.A., et al., Sequences of Proteins of Immunological Interest, 5th ed., Public Health Service, National Institutes of Health, Bethesda, MD (1991)) as defined above.

### EXAMPLES

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer’s instructions. General information regarding the nucleotide sequences of human immunoglobulin light and heavy chains is given in: Kabat, E.A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning / sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Cell Culture Techniques

Standard cell culture techniques were used as described in Current Protocols in Cell Biology (2000), Bonifacino, J.S., Dasso, M., Harford, J.B., Lippincott-Schwartz, J. and Yamada, K.M. (eds.), John Wiley & Sons, Inc.

All genes are transiently expressed under control of a chimeric MPSV promoter consisting of the MPSV core promoter combined with the CMV promoter enhancer fragment. The expression cassette also contains a synthetic polyA signal at the 3′ end of the cDNAs. The expression vector also contains the oriP region for episomal replication in EBNA (Epstein Barr Virus Nuclear Antigen) containing host cells.

### Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at -20° C. or -80° C. Part of the samples were provided for subsequent protein analytics and analytical characterization e.g. by SDS-PAGE, size exclusion chromatography (SEC) or mass spectrometry.

### SDS-Page

The NuPAGE® Pre-Cast gel system (Invitrogen) was used according to the manufacturer’s instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

### Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2 × PBS on a Dionex HPLC-System. The eluted protein was quantified by UV absorbance and integration of peak areas. BioRad Gel Filtration Standard 151-1901 served as a standard.

### Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 µg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

### Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3 - 10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

### Preparation, Purification and Characterization of Bispecific Antigen Binding Molecules Targeting Fibroblast Activation Protein (FAP) and CD40

Bispecific antigen binding molecules targeting fibroblast activation protein (FAP) and CD40 were prepared as described in International Patent Appl. Publ. No. WO 2018/185045 A1, WO 2020/070041 A1 or WO 2020/070035 A1.

In particular, the following molecules were made:

a) Bispecific antigen binding molecules targeting fibroblast activation protein (FAP) and CD40

Bispecific FAP-CD40 antibodies were prepared in 2+1 format consisting of two CD40 binding moieties combined with one FAP binding moiety at the C-terminus of an Fc (FIG. 1D) or in 4+1 format consisting of four CD40 binding moieties combined with one FAP binding moiety at the C-terminus of an Fc (FIGS. 1A or 1B). The bispecific CD40-FAP antibodies included anti-FAP clone 212 as disclosed in WO 2020/070041 A1 or FAP clones 4B9 and 28H1 which have been described in WO 2012/020006 A2. To generate the 4+1 and the 2+1 molecules the knob-into-hole technology was used to achieve heterodimerization. The S354C/T366W mutations were introduced in the first heavy chain HCl (Fc knob heavy chain) and the Y349C/T366S/L368A/Y407V mutations were introduced in the second heavy chain HC2 (Fc hole heavy chain). Independent of the bispecific format, for hu IgG1 an effector silent Fc (P329G; L234, 234A) was used to abrogate binding to Fcy receptors according to the method described in WO 2012/130831 A1. Sequences of the bispecific molecules are shown in Table 1. As control molecules, untargeted versions were prepared accordingly by replacing the FAP clone by germline DP47.

TABLE 1

FAP-CD40 bispecific antibodies

| | |
|---|---|
| Construct | composed of |
| CD40 (P1AE0817) x FAP (P1AE1689) 2+1 bispecific antibody (C-terminal crossfab with VH/VL crossing) | 1 x LC of SEQ ID NO:61, 2 x LC of SEQ ID NO:62 1 x HC of SEQ ID NO:63 and 1x HC of SEQ ID NO:64 |
| CD40 (P1AE0817) x FAP (P1AE1689) 4+1 bispecific antibody (C-terminal crossfab with VH/VL crossing) | 1 x LC of SEQ ID NO:61, 4 x LC of SEQ ID NO:62 1 x HC of SEQ ID NO:65 and 1x HC of SEQ ID NO:66 |
| CD40 (P1AE0817) x FAP (28H1) 2+1 bispecific antibody (C-terminal crossfab with VH/VL crossing) | 1 x LC of SEQ ID NO:67, 2 x LC of SEQ ID NO:62 1 x HC of SEQ ID NO:68 and 1x HC of SEQ ID NO:64 |

b) Murine Surrogates and Control Molecules

In a similar way, bispecific FAP-CD40 antibodies comprising FAP clone 28H1 and a CD40 clone binding to murine CD40 (FGK4.5) were prepared for use in studies with mice. The molecules were prepared in 2+1 format consisting of two CD40 binding moieties combined with one FAP binding moiety at the C-terminus of an Fc (FIG. 1D) or in 4+1 format consisting of four CD40 binding moieties combined with one FAP binding moiety (VH and VL connected at C-terminus of an Fc, FIG. 1B). Sequences of these bispecific molecules are shown in Table 2. As control molecules, untargeted versions were prepared accordingly by replacing the FAP clone by germline DP47.

TABLE 2

Murine Surrogates and Control molecules

| | |
|---|---|
| Construct | composed of |
| muCD40 x FAP (28H1) 4+1 bispecific antibody (VH on knob and VL on hole) (P1AD9139) | 4 x LC of SEQ ID NO:69 1 x HC of SEQ ID NO:70 and 1 x HC of SEQ ID NO:71 |
| muCD40 x DP47 4+1 bispecific antibody (VH on knob and VL on hole) (P1AE2425) | 4 x LC of SEQ ID NO:69 1 x HC of SEQ ID NO:72 and 1 x HC of SEQ ID NO:73 |
| CD40 (P1AE0817) x FAP (28H1) 2+1 mu IgG1 DAPG bispecific antibody (C-terminal crossfab with VH/VL crossing) (P1AE2302) | 1 x LC of SEQ ID NO:74, 2 x LC of SEQ ID NO:75 1 x HC of SEQ ID NO:76 and 1x HC of SEQ ID NO:77 |

### Example 2

### In Vivo Anti-Tumor Efficacy of FAP-Targeted Anti-CD40 Antigen Binding Molecules in Combination With Radiotherapy as Shown in the mEERL95 Tumor Model

### 2.1 Material and Methods

The ability of bispecific FAP-targeted anti-CD40 antigen binding molecules to achieve therapeutic efficacy either alone or in combination with a hypofractionated radiation therapy regimen was tested in mice bearing the mEERL95 tumor cell line, a clinically relevant mouse model for HPV-related head and neck squamous cell carcinoma (HNSCC).

Seven- to eleven-week-old female C57BL/6J mice were purchased from Janvier Labs. Humanized hCD40Tg mice were provided by Roche Glycart AG (Zurich). Batf3-/- mice were kindly gifted by Dr. Hans Acha Orbea (University of Lausanne) and bred in the facilities of University of Lausanne. All animal procedures were carried out under the license VD3173j which was approved by the Veterinary Authority of the Swiss Canton of Vaud.

The mEERL95 cell line was derived from a tumor explant of a mEERL-bearing C57BL/6J female (Mermod et al., Int. J. Cancer 2018, 142, 2518-2528) and cultured in DMEM/nutrient mixture F-12 (Thermo Fisher Scientific) medium supplemented with 5% fetal bovine serum (FBS, Life Technologies) and 1 x human keratinocyte growth supplement (HKGS, Thermo Fisher Scientific). TC-1 cells (Lin et al., Cancer Research 1996, 56, 21-26) were kindly provided by T.C. Wu (Johns Hopkins) in 2009 and grown in RPMI 1640 medium (Life Technologies) supplemented with 10% FBS (Life Technologies), penicillin/streptomycin (Life Technologies) and 5 × 10⁻⁵ M 2-mercaptoethanol (Life Technologies). Both cell lines were cultured in an incubator at 37° C. and 5% CO₂ and routinely tested to discard mycoplasma. Cells were harvested for inoculation into mice when reached 90% of confluence.

### Tumor Model and In Vivo Treatments

Mice were subcutaneously injected in the submental region with 1×10⁵ mEERL95 or TC1 cells resuspended in 30 µL of HBSS (Hank’s Balanced Salt Solution, Thermo Fisher Scientific) and 20 µL of Matrigel (Corning). Tumors were irradiated with a hypofractionated regimen consisting in two consecutive doses of 6 Gy (2× 6 Gy) at day 10 and 11 post-tumor engraftment. Doses were delivered with an Xrad-225CX-PXi Instrument using a 15-mm collimator that allows to locally irradiate the tumors.

At day 11, mice were treated with a single i.p injection of FAP-targeted bispecific anti-muCD40 antibody (FAP-CD40) or the corresponding untargeted anti-CD40 control antibody (DP47-CD40) at 18.3 mg/kg and 10 mg/kg in PBS, respectively. mEERL95-bearing huCD40Tg mice were irradiated with the 2× 6 Gy regimen and treated with a single i.p dose of FAP-huCD40 at 13 mg/kg in PBS. Tumor size was followed up using a caliper twice per week and the volume was calculated with the following formula:

V = (length x width²)/2 (V = Tumor volume)

For the depletion studies, 200 µg/dose of anti-CD8β mAb (clone H35-17-2), anti-CD4 (clone GK1.5) or a Rat IgG2a (clone 2A3, BioXCell), used as immunoglobulin isotype control, were given to mEERL95-bearing mice on days -2 and 0 with respect to the day of treatment initiation. The administration of the antibodies was done every three days for two weeks. The efficiency of the depletion was checked by flow cytometry at day 18 on peripheral blood lymphocyte. For the in vivo experiments blocking IL-12, mice were daily treated with 500 µg of anti-IL-12 (clone C17.8, BioXCell) diluted in PBS for one week starting the same day of the therapeutic antibody administration. Same doses of a Rat IgG2a (clone 2A3, BioXCell) were used as immunoglobulin isotype control.

### Tumor Rechallenge

In order to characterize the development of immunologic memory, 10⁶ mEERL95 or 10⁵ TC-1 tumor cells resuspended in PBS were subcutaneously engrafted in the flank of mice that had rejected the primary tumors following the therapy. Tumor inoculations were performed approximately 40 days after developing complete response. Tumor growth was followed up by measuring every 2-3 days with a caliper.

### Cell Isolation

Tumors and regional lymph nodes were harvested 10, 16 or 18 days post tumor engraftment. Collected tumors and lymph nodes were incubated at 37° C. with 1 mg/ml of Collagenase-D and 40 µg/ml DNase-I (Sigma-Aldrich) for 45 and 20 min, respectively. Afterwards, they were mechanically disaggregated and filtrated using a 70-µm cell strainer (Falcon, BD Bioscience) in order to obtain single-cell suspensions. Additionally, tumor-infiltrating lymphocytes were isolated from stromal cells with a 35% Percoll gradient. When needed, red blood cells were lysed for 3 min at RT with RBC buffer (Qiagen) before flow cytometry staining.

### Flow Cytometry

Single-cell suspensions were firstly incubated with FcR-Block (anti-CD16/32 clone 2.4G2, homemade) for 15 min on ice in FACS buffer (2% FCS and 2 mM EDTA in PBS). To characterize the immune populations, samples were surface stained with the following antibody panel in darkness for 20 min on ice: CD1 1c-BV421(clone N418), CD8-BV510 (clone 53-6.7), F4/80-BV605 (clone BM8), NK1.1-BV650 (clone PK136), CD11b-BV711 (clone M1/70), CD4-BV785 (clone RM4-5), CD19-BV785 (clone 6D5), Ly6G-FITC (clone 1A8), CD45-PerCP (clone 30-F11), Ly6C-AF700 (clone HK1.4), IA/IE-APC-Cy7 (clone M5/114.15.2) and B220-APC (clone RA3-B2) from Biolegend; CD80-Pe-Vio770 (clone 16-10A) from Miltenyi Biotec; CD103-PE (clone 2E7), CD3-PE-Cy5.5 (clone 145-2C11) and Foxp3-PeFluor-610 (clone FJK-165) from eBioscience. Intracellular staining of Foxp3 was performed with the Foxp3/Transcription Factor Staining Buffer Set (eBioscience) according to the manufacturer’s instructions. LIVE/DEAD™ Fixable Blue Dead Cell Stain Kit (Thermo Fisher Scientific) was used as a viability marker.

For the ex-vivo stimulation assays, lymphocytes from tumor and lymph nodes were co-cultured with mEERL95 cells in a 10:1 ratio. The mEERL95 cells were previously incubated with IFN-γ (200 ng/ml; ImmunoTools) for 24 h. The co-culture was done in presence of 1 µg/ml of anti-CD28 (clone 37.51) and 10 µg/ml of anti-PD1 (clone RMP1-14, BioXCell), and kept for 16 h at 37° C. in complete medium. Unstimulated cells were used as negative controls. GolgiPlug and GolgiStop (both 1:1000; BD Biosciences) were added to the cells 4 h before starting the staining. Upon surface staining with CD45-BV650 (clone 104, BioLegend), CD3- (clone 17A2, eBioscience), CD8- (clone 53-6.7, eBioscience), CD4-(clone GK1.5, BioLegend), cells were fixed and permeabilized with a fixation buffer (BioLegend) for 20 mins on ice. Afterwards, cell suspensions were stained with the following intracellular antibodies diluted in Perm/wash buffer 1x (BioLegend): IFNγ-PerCp-Cy5.5 (clone XMG1.2, eBioscience), TNFα-PB (clone MP6-XT22, BioLegend) and Granzyme B-PerCp-Cy5.5 (clone QA16A02, BioLegend) for 30 mins on ice. Samples were acquired either with LSRII-SORP and Fortessa flow cytometers (BD Biosciences). Data analyses were performed using FlowJo v10 (FlowJo LLC).

### Immunofluorescence and Imaging

Tumors pieces were embedded in Cellpath™ OCT cryo microtomy embedding matrix (Cell Path Ltd. Newton, Powys UK) and frozen on dry ice. Ten-micrometer frozen sections were fixed in 2% paraformaldehyde (PFA, Sigma) for 10 min and washed three times with phosphate-buffered saline (PBS). Primary antibodies were incubated in PBS containing 0.5% of bovine serum albumin (BSA, Sigma), 0.3% of Triton X-100 (AppliChem GmbH Germany) and 1% of serum from the same host species of the secondary antibodies. Slides were kept in a humid chamber at 4° C. overnight. After three washes with PBS, samples were stained for one hour at RT with secondary antibodies. Tissue slices were washed three additional times with PBS, and mounted with Prolong™ Gold anti-fade reagent containing 4′,6-diamidino-2-phenylindole (DAPI). Following immunofluorescence, slides were digitalized on a ZEISS Axio Scan Z1. Downstream analysis was performed with FIJI, an open-source platform for biological image analysis (Schindelin et al., Nature Methods 2012, 9, 676-682) using custom-made scripts. Briefly, DAPI staining was used to estimate the total area of the section while E-cadherin staining was used to measure the tumor area. For the quantification of well-delimited cells such as immune cells, an infiltration index was calculated based on the count of positive cells detected per area analyzed. For non-discernable group of cells such as mesenchymal cells, the quantification was performed by computing the ratio between the size of the area positive for the immunostaining divided by the total size of the area analyzed.

### Immunohistochemistry

Lymph nodes were collected and fixed in 1% PFA overnight at 4° C., washed two times with PBS and incubated in 30% sucrose solution for 3-6 h at 4° C. Afterwards, lymph nodes were embedded in OCT embedding matrix (Cell Path Ltd. Newton, Powys UK) and frozen on dry ice. Eight-micrometer frozen sections were directly incubated with the primary antibody against mouse FAP (clone 4B9, Roche Glycart AG) for 16 h at 4° C. Upon washing, samples were incubated with a biotin-conjugated goat anti-rabbit as secondary antibody and revealed with the Vectastin ABS kit (Vector labs).

### Multiplex Assays

Cytokine and chemokine concentration from the sera and tumors was measured with the LegendPlex™ Mouse Proinflammatory Chemokine Panel and LegendPlex™ Mouse Inflammation panel (both from Biolegend) following Manufacturer’s instructions. Analysis was performed using LegendPlex™ data analysis software (Biolegend, San Diego CA).

### RNA Extraction, Real Time and Gene Expression Analysis

Total RNA from tumor pieces was extracted using DirectZol™ RN A MiniPrep Kit (ZymoResearch). Reverse transcription was performed with M-MLV reverse transcriptase (Invitrogen) and quantitative real time PCRs (q-PCR) were carried out with Fast SyberGreen PCR Master Mix (Thermo Fisher Scientific) on an ABI Prism 7500 fast device (Thermo Fisher Scientific). qPCRs were carried out with primers for mouse Fap cDNA (fw: 5′-GTCACCTGATCGGCAATTTGT -3′, rev: 5′- CCCCATTCTGAAGGTCGTAGAT -3′) which expression was normalized with the mouse Tbp levels (fw: 5′-CCTTGTACCCTTCACCAATGAC -3′, rev: 5′-ACAGCCAAGATTCACGGTAGA -3′). The expression was represented according to this formula:

2 ^{ΔCt} (^{Ct} ^{tbp} ⁻ ^{Ctfap}), where Ct corresponds to cycle number.

In order to study the expression of immune-related genes in the tumor microenvironment, 100 ng of total RNA extracted from tumors at day 14 was hybridized to the nCounter® PanCancer immune profiling panel (NanoString Technologies). Gene counts were normalized with the selected housekeeping genes from the panel using the nSolver™ 2.6 software (NanoString Technologies, Seattle).

### Statistical Analysis

Differences in immune cell frequencies and tumor growth and mice survival were analyzed with GraphPad Prism (GraphPad Software, La Jolla, CA). T-test, ANOVA for multiple comparison or the corresponding nonparametric tests (Mann-Whitney or Kruskal-Wallis) were used. Long-rank test was used for survival analysis. P values <0.05 were considered significant.

### 2.2 Results

FAP-CD40 in combination with RT combination therapy is safe, efficient and requires crosslinking via FAP to mediate control of tumor growth

To test the therapeutic potential of the FAP-targeted CD40 bispecific antibody (FAP-CD40), an orthotopic head and neck tumor model (mEERL95) was used that has been previously generated (Mermod et al., Int. J. Cancer 2018, 142, 2518-2528) and that recapitulates key clinical aspects of the disease, including a FAP-positive stroma. In a first experiment, the anti-tumor efficacy and safety of FAP-CD40 either alone or combined with hypofractionated radiotherapy (consisting of two consecutive doses of 6 Gy) or not combined was investigated according to the scheme shown in FIG. 2A. The untargeted DP47-CD40 control bispecific antibody (DP47 is a germline control) showed no anti-tumor effect in tumor-bearing mice as did the PBS-only treatment (FIGS. 2C and 2D). On the contrary, FAP-CD40 led to complete tumor regression in 40% of treated mice followed by tumor relapse in one mouse (FIG. 2E). These results indicate that FAP-CD40 requires crosslinking via FAP to be therapeutically efficient.

Local radiotherapy, given alone (FIG. 2F) or together with the control DP47-CD40 antibody (FIG. 2G), resulted in tumor growth delay and increased survival as compared with control groups, i.e. PBS-untreated and DP47-CD40-treated mice (FIGS. 2C and 2D). However, from day 20 post mEERL95 tumor implantation, 2 × 6 Gy and 2 × 6 Gy + DP47-CD40-treated tumors started to relapse thus resulting in no objective complete responses (CR). Interestingly, all tumor-bearing mice responded to the combination of radiotherapy (2 × 6 Gy) with FAP-CD40 antibody, which induced complete tumor regressions and durable control in 83% (⅚) of the animals (FIG. 2H). An additional dose of anti-FAP-CD40 at day 13 did not improve the outcome of the combination (FIGS. 2K and 2L). Overall, with 2 out of 5 CR, mice treated with FAP-CD40 as single agent, significantly increased the overall survival as compared to PBS and DP47-CD40 control groups (FIG. 2B). Remarkably, all responder mice from the combination treatment remained relapse-free after prolonged periods of observation (more than 90 days) showing that this FAP-CD40 radioimmunotherapy combination not only induced a high tumor regression rate but also extended animal survival (FIG. 2B).

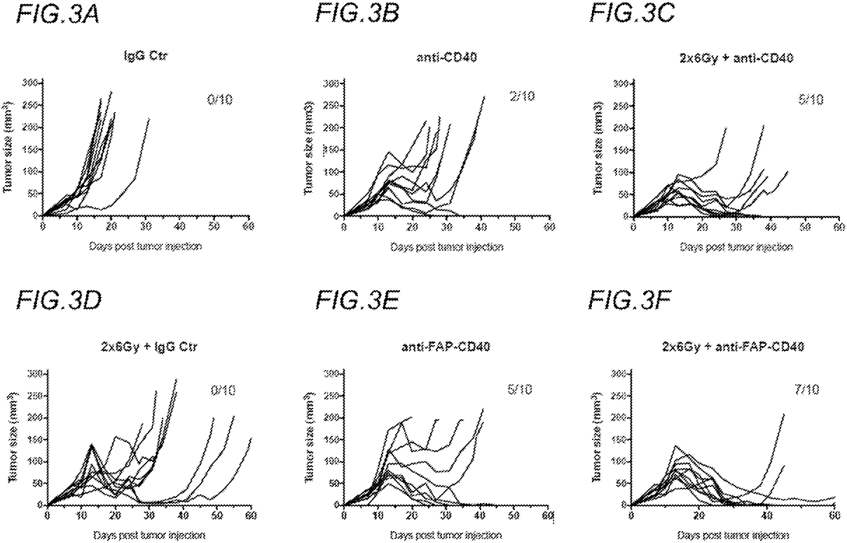

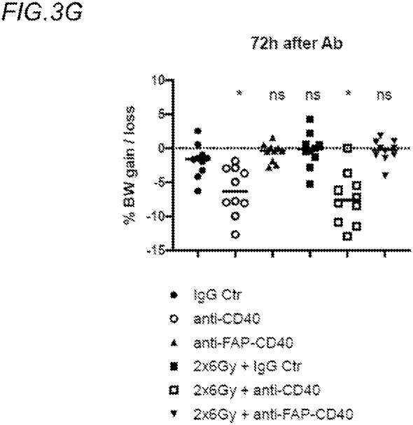

The efficacy of the FAP-CD40 antibody as a single agent or in combination with hypofractionated radiotherapy was accompanied with absence of systemic toxicity, a common feature observed in the therapy with CD40 agonistic monoclonal antibodies (Vonderheide et al., J. Clin. Oncol. 2007, 25(7), 876-883; Medina-Echeverz et al., Cancer Immunol. Res. 2015, 3(5), 557-566, or Vonderheide et al., Oncoimmunology 2013, 2(1):e23033). In a second comparative experiment, tumor-bearing mice treated with the FAP-CD40 antibody did not present body weight loss whereas a single injection of a CD40 mAb (of the same molarity), combined or not with RT, induced a significant loss of body weight with tumor growth control comparable to that mediated by the bispecific FAP-CD40 antibody therapy. The results of the second experiment are illustrated in FIGS. 3A to 3F, in FIG. 3G the corresponding change in body weight is shown.

### Presence of FAP-Expressingfibroblasts in the Tumor Stroma is Required for an Efficient FAP-CD40 Activity

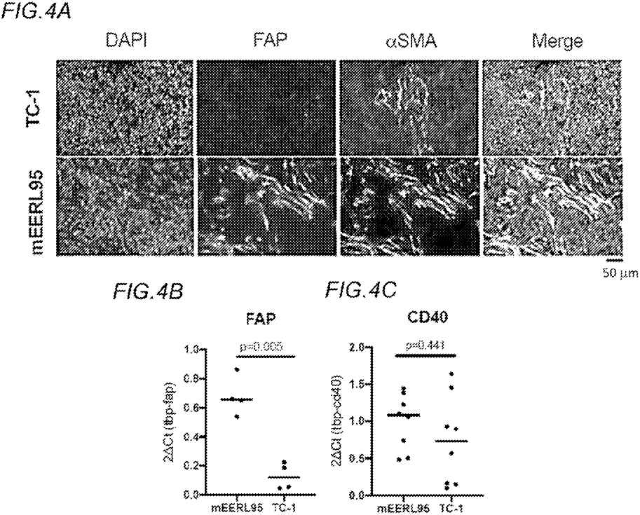

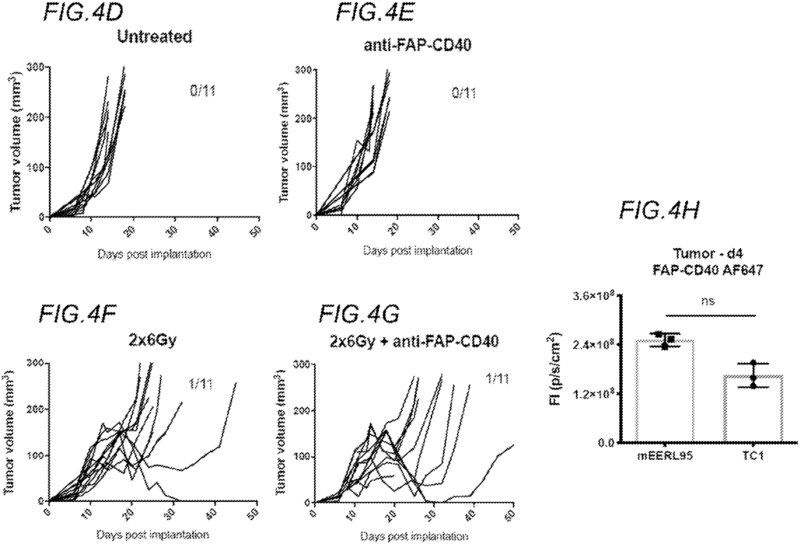

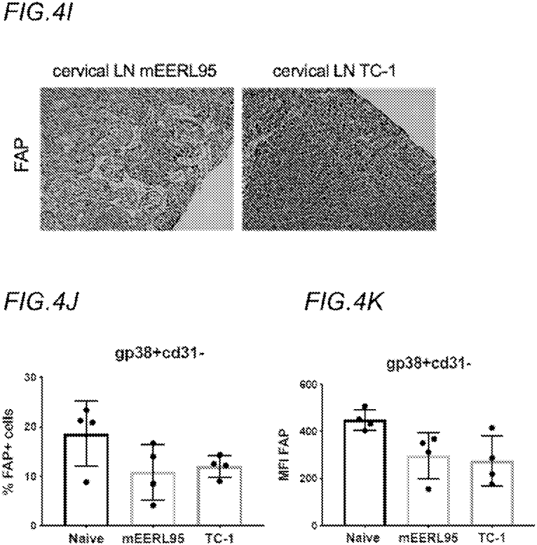

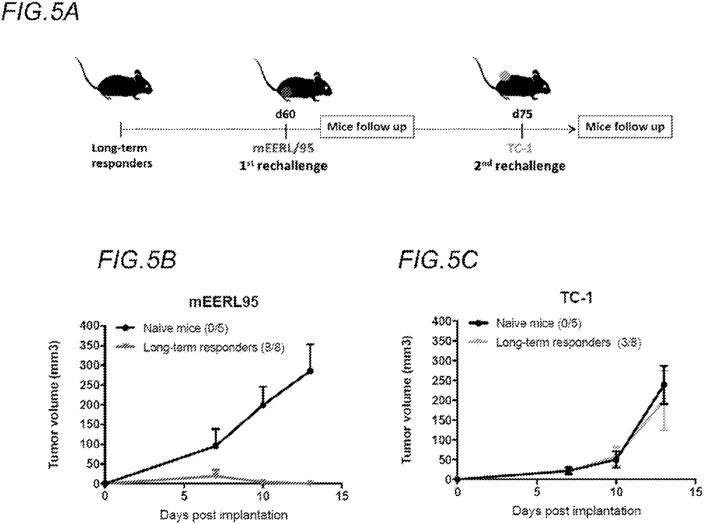

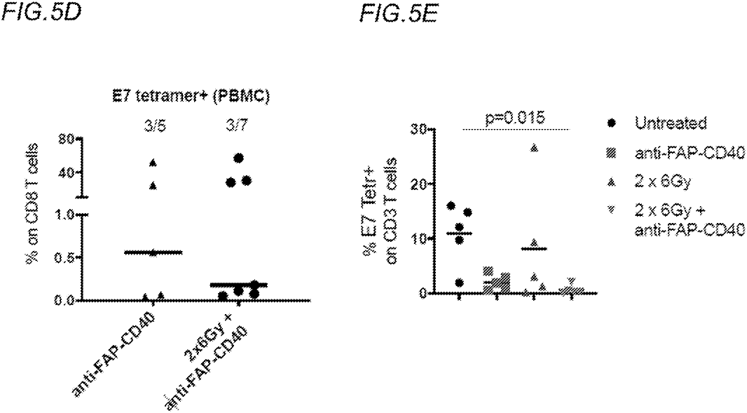

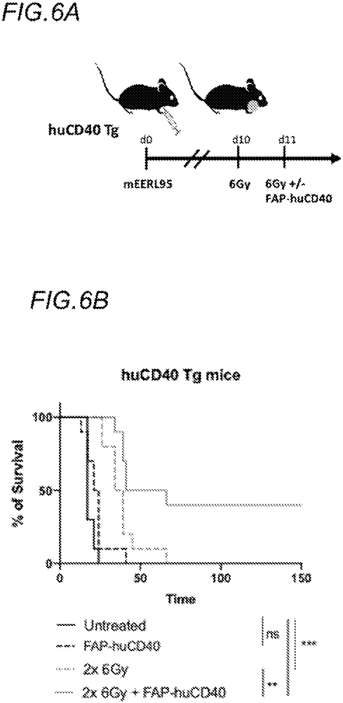

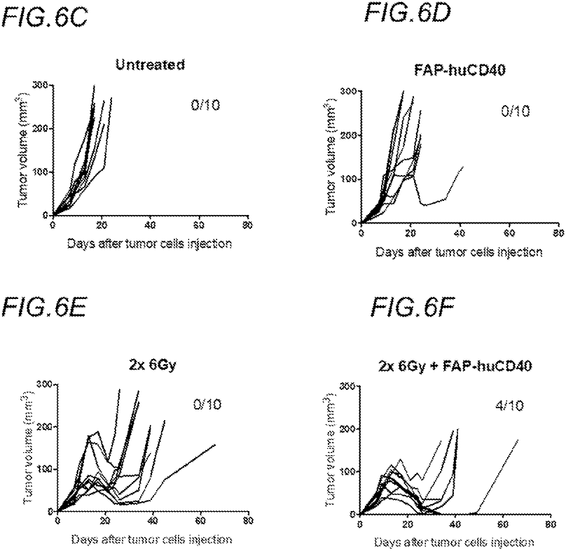

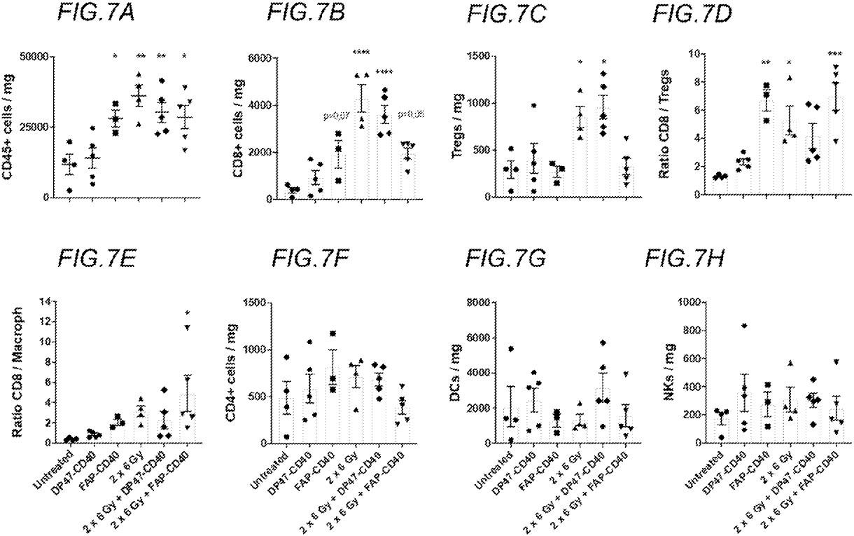

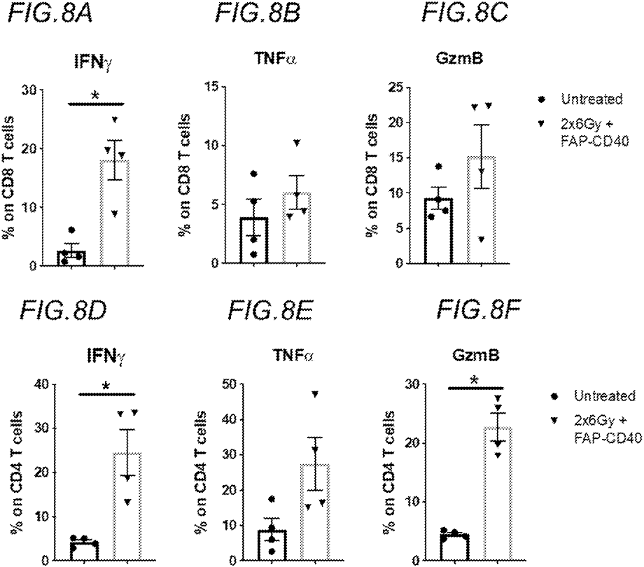

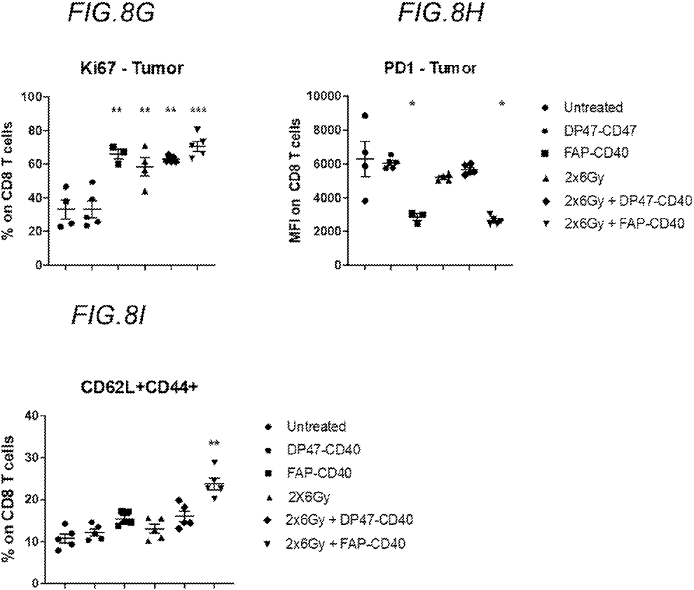

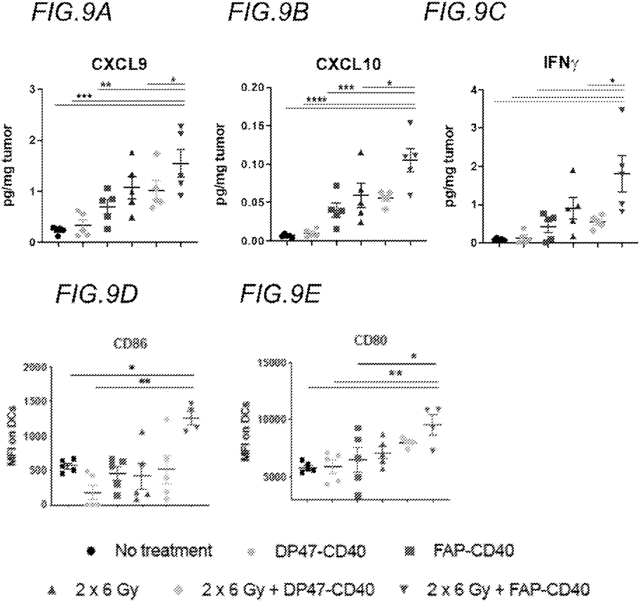

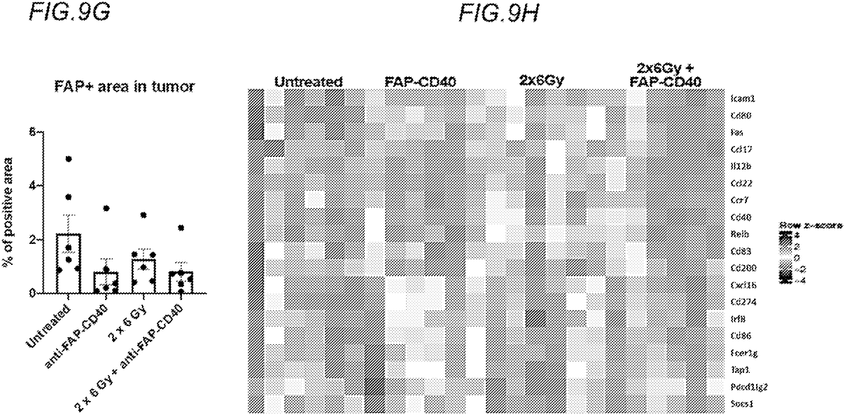

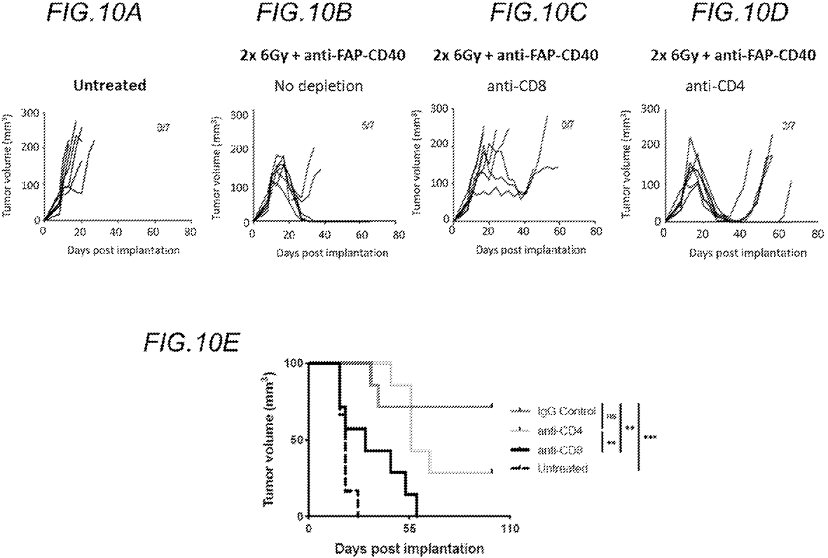

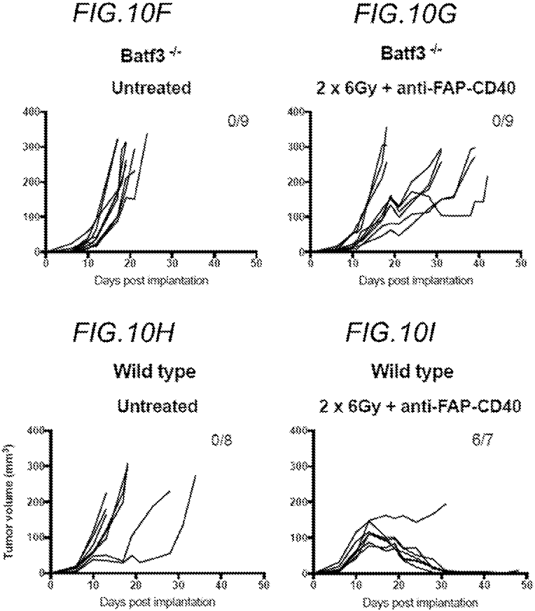

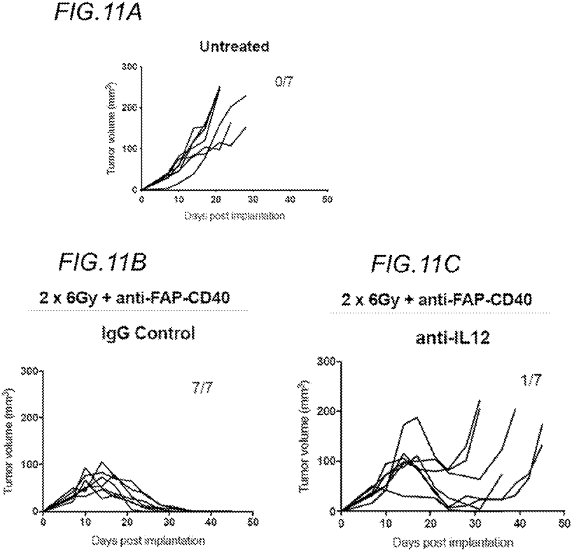

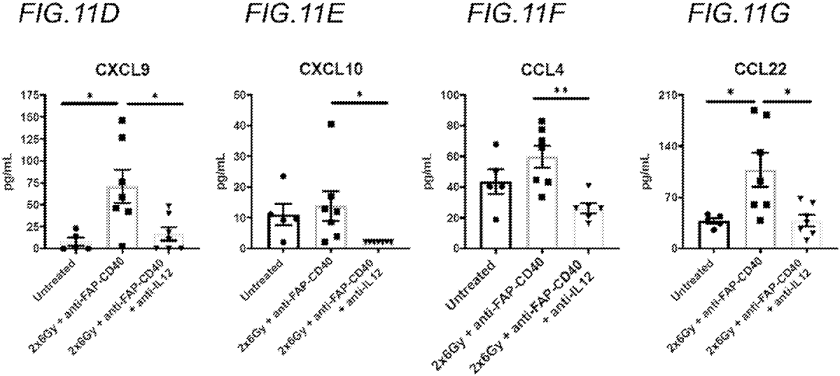

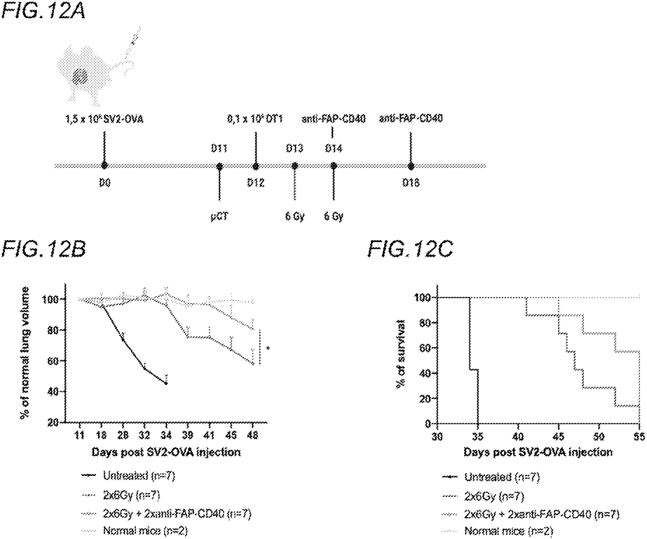

FAP, a prolyl endopeptidase, is highly expressed in cancer-associated fibroblasts (CAFs) from human HNSCC and recapitulated in the mEERL95 model as shown in FIG. 4A. To investigate further whether FAP-CD40-mediated anti-tumor activity required FAP-expressing stromal fibroblasts, mouse TC-1 lung cancer cells expressing mutant H-ras, HPV16 E6 and E7 proteins (Lin et al., Cancer Research 1996, 56, 21-26) but devoid of FAP expression (Dupperet et al., Clin. Cancer Res. 2018, 24(5), 1190-1201) were used. TC-1 tumor cells implanted in the submental area resulted in tumors with peritumoral αSMA-expressing CAFs and a few intratumoral areas of αSMA-positive, FAP-negative cells (FIGS. 4A and 4B). In line with the results of immunostaining, mRNA expression level of Fap gene is significantly lower in the TC-1 tumors (FIG. 4B).

Consistently, and unlike the outcome observed in the mEERL95 model, TC-1 tumors did not respond to FAP-CD40 alone and showed only 9% (1/11) of complete responses (CR) upon the treatment with either RT alone or the combination of 2× 6 Gy and FAP-CD40 (FIGS. 4D to 4G). To assess FAP-CD40 antibody access to the TC-1 tumor, we intraperitoneally injected the anti-FAP-CD40 antibody labeled with Alexa647 into TC-1 and mEERL95 tumor-bearing mice that underwent local irradiation. The antibody was detectable in both tumors four days upon administration. No significant difference in the fluorescence intensity was detected in both TC-1 and mEERL95 tumors (FIG. 4H) which present similar CD40 mRNA expression levels (FIG. 4C). These results indicate that FAP-CD40 is indeed able to target the CD40-expressing cells present in the TC-1 tumor microenvironment but fail to promote protective immunity as no activation of CD40 signaling can occur in the absence of FAP-mediated cross-linking.

As FAP is also present in the lymph node stromal compartment, especially in the fibroblastic reticular cells (FRCs) (Denton et al., Proc. Natl. Acad. Sci. USA 2014, 111(33), 12139-12144), the levels of FAP in the regional lymph nodes from the two tumor models were examined. FAP expression was detected by IHC in the cervical lymph nodes from both mEERL95 and TC-1 submental tumors at 10 days post inoculation (FIG. 4I). The frequency and the expression of FAP on FRC (gp38+CD31-) measured by flow cytometry was also similar in TC-1 and mEERL95-bearing mice (FIGS. 4J and 4K). These results emphasize the requirement of FAP expression in the tumor stroma.

### FAP-CD40 Combination With Radiotherapy Promotes Long-Term Anti-Tumor Protective Immunity

In order to determine whether the combination of radiotherapy (2 × 6 Gy) with anti-FAP-CD40 treatment was able to induce tumor-specific long-term protective immunological memory in mEERL95 tumor-bearing mice, those mice that rejected mEERL95 primary tumors upon the combination (long-term responders) were re-challenged with the homologous tumor in the right flank (FIG. 5A). All mice controlled tumor growth and eventually rejected them, indicating the existence of specific immune memory (FIG. 5B). As mEERL95 cells express the HPV16 E7 protein, we hypothesized that such a protein could be the immunodominant target antigen of the immunological memory observed. To test this hypothesis, cured mice were exposed to a second re-challenge with the TC-1 cell line, which also expresses the E7 protein, in the left flank (FIG. 5A). Only 3 out of 8 mice were able to reject TC1 cells suggesting that E7 may not be the immunodominant antigen in this context (FIG. 5C). In addition, around 40-60% of long-term responder mice (which rejected the mEERL95 primary tumor upon treatment with either FAP-CD40 as single agent or combined with RT) presented E7-specific T lymphocytes upon in vitro expansion of PBMCs (FIG. 5D). In fact, the frequency of E7-specific CD8 T cells (detected by specific tetramer staining) decreases in the mEERL95 tumor microenvironment upon the combination as compared to untreated tumors (FIG. 5E).

Prompted by the encouraging results and with the aim of promoting the translation of this combination therapy to the clinic, the antitumor properties of a surrogate bispecific antibody that is composed of the anti-mouse FAP moiety but targets the human CD40 receptor (FAP-huCD40) was tested in a further experiment. Therefore, mEERL95 tumor cells were inoculated in the submental region of transgenic mice expressing the human CD40 receptor (huCD40 Tg mice). Following the same schedule of treatment as in wild-type tumor bearing-mice, the tumors were locally irradiated with two consecutive doses of 6 Gy. Afterwards, mice were administered an i.p. injection of the FAP-huCD40 antibody coinciding with the second radiotherapy session (FIG. 6A). With no complete responses observed, FAP-huCD40 as a single agent did not differ in tumor growth or survival to untreated mice (FIGS. 6B and 6D). Mice that underwent 2×6 Gy slowed down the tumor growth, induced tumor regression and increased the survival compared to FAP-CD40 and control mice; however, none of them completely rejected the tumors (FIGS. 6B and 6E). The combination of 2×6 Gy and FAP-huCD40 resulted in 40% of complete responses (4/10) and significantly prolonging long-term survival in comparison to the rest of the groups (FIGS. 6F and 6B).

### Combination of FAP-CD40 With Radiotherapy Remodels the Tumor Immune Microenvironment Into a Less Immunosuppressive Landscape

Based on the first experiment (FIG. 2A), the cellular and molecular immune components associated with the anti-tumor effect of the combination were explored. The intra-tumoral immune infiltrate was analyzed eight days following the first radiotherapy dose. Using a flow cytometry based 16-color antibody panel, an overall increase in the number of immune cells (CD45+ cells) per mg of tumor was observed in all treated groups compared to PBS and DP47-CD40 (FIG. 7A). Compared to PBS and DP47-CD40 control, the number of CD8 T cells increased significantly upon irradiation. This increase was less pronounced upon FAP-CD40 monotherapy and combination treatment (FIG. 7B). The number of regulatory T cells (Tregs) present in the tumor was only increased upon radiotherapy as compared to control mice (FIG. 7C), giving rise to an increase in the CD8/Tregs ratio in FAP-CD40, RT and combined treatment groups (FIG. 7D). Interestingly, CD8 cells/macrophages ratio increases only in the FAP-CD40 + 2×6 Gy group supporting the idea that the combination may shift the balance in favor of CD8 T cells infiltration in the tumor (FIG. 7E). In this context, although the frequencies of CD4 T cells (FIG. 7F), dendritic cells (DCs, FIG. 7G) and NKs (FIG. 7H) decreased with the different treatments compared to the untreated mice, the numbers per mg of tumor were comparable among all of the groups.

Furthermore, an increase in the production of IFNγ by CD8 and CD4 T cells from tumor-infiltrating lymphocytes (TILs) from tumors treated with FAP-CD40 and RT was observed in ex-vivo re-stimulation assays against mEERL95 cells (FIGS. 8A and 8D). TNFα and Granzyme B were also augmented in both T cell subsets (FIGS. 8B, 8C, 8E and 8F) indicating an effector and cytotoxic phenotype The proliferation of CD8 TILs, measured as Ki67 positive cells (FIG. 8G), is increased in all treated groups compared to the controls, whereas the expression of PD-1 remains lower in a FAP-CD40 dependent manner suggesting a less exhausted phenotype (FIG. 8H). The combination therapy promoted a memory phenotype (CD62L⁺CD44⁺) on CD8 T cells from the regional lymph nodes (FIG. 8I).

The combination therapy significantly amplified the molecular remodeling of the tumor microenvironment by upregulating 399 and downregulating 39 immune-related genes as compared to untreated tumors. This molecular imprint triggered by the combination therapy encompassed an induction in the expression of different pathways involved in adaptive immune response, antigen processing, interferon signaling, inflammation, chemokines and cytokines or DCs functions. A significant enrichment in a dendritic cell-specific inflammatory gene signature was found in tumors that underwent the combination therapy of 2× 6 Gy and FAP-CD40 treatment that includes Cd40, Ccl22, Il12b, Irf8 and Ccr7 upregulation. By multiplex cytokine/chemokine assay, upregulation of intratumoral levels of IFNγ levels and certain chemokines such as CXCL9 and CXCL10 was confirmed that was triggered by the combination therapy (FIGS. 9A, 9B and 9C). We also found an induction of maturation markers such as CD80 and CD86 on the surface of DCs from mEERL95 infiltrate upon 2×6Gy+FAP-CD40 (FIGS. 9E and 9F).

Altogether, these data demonstrate that the remarkable efficacy of the combination of FAP-CD40 therapy and RT with 2× 6 Gy is associated with major remodeling of the tumor immune landscape including reduced immunosuppression, increased CD8 T cell infiltration and proliferation, cytokine production and maturation of DCs.

### The Therapeutic Efficacy of the Combination is Dependent on CD8 T Cells and Cross-Priming DCs, and Relies on CD4 T Cells for Relapse Prevention

By in vivo selective depletion the contribution of T lymphocytes to the anti-tumor effect of the combination therapy was examined. Forty-eight hours before initiating the therapy, mEERL95 tumor-bearing mice received i.p. injections of either anti-CD8β, anti-CD4 or the respective IgG control antibody every three days starting 48 h before the therapeutic treatment. The depletion treatment of mice with CD8 T lymphocyte-targeted antibodies resulted in an abrogation of the curative effect observed upon the combination therapy (FIG. 10C). Mice depleted of CD4 T cells showed complete responses by day 30 post-treatment (FIG. 10D). However, 2 out of 7 (28%) mice from this group underwent tumor relapses as compared to the 5 out of 7 (71%) mice from the IgG control group (FIG. 10B) resulting in reduced survival (FIG. 10E). These results highlight an essential role of CD4 T cells in formation of a durable anti-tumor response.

The enrichment of DCs maturation transcriptional signature in the tumors upon combination therapy, suggested a role of activated Batf3-dependent DCs and the necessity for CD8 T cells-cDC1 crosstalk in CD8 T cell-mediated responses. We therefore sought to test the combination therapy in the absence of such an immune population. As FIGS. 10F to 10I show, the therapeutic effect of the combination of RT (2×6 Gy) and FAP-CD40 treatment was abolished in Baif3-/- mice (which lack cDC1). Although tumor growth kinetics decreased in some treated Baif3-/- mice as compared to non-treated tumor-bearing Batf3-/- mice, the anti-tumor effect of the combination therapy was abrogated as opposed to treated tumor-bearing wild-type mice, indicating the requirement of cross-priming DCs for the FAP-CD40-mediated robust protective response.

### Blocking IL-12 During the T Cell Effector Phase Abrogates the Efficacy of the Combination Therapy

Given that IL12b is one the top differentially tumor expressed genes upon the combination therapy and known cytolytic activity of IL-12 its functional relevance in the therapeutic response to the combination of RT (2× 6 Gy) and FAP-CD40 was studied. Thus, an IL-12 blocking antibody was administered for seven days starting one day before radiotherapy. As FIG. 11C shows, in all mice tumors regressed upon combination therapy and IL-12 neutralization, but tumors resumed growth in 85% mice (6 out 7), thus strongly supporting the key role of IL-12 in attaining the full strength of the anti-tumor effect mediated by combination therapy. Interestingly, the blocking of IL-12 does not affect the response at early stages when anti-tumor activity is likely mainly due to radiotherapy. The loss of the therapeutic efficacy upon IL-12 neutralization was accompanied by a reduction in the serum levels of certain chemokines produced by dendritic cells such as CXCL9, CXCL10, CCL4 and CCL22 (FIGS. 11D to 11G), which are associated with T-cell recruitment, migration and priming.

### 2.3 Discussion of Results

It has been shown that the combination of a bispecific FAP-CD40 antibody with RT is a novel safe and efficacious strategy to enhance radiation therapy and achieve durabable protective immune memory in HPV+ HNSCC. The key component of this combination is based on limiting the activity of a CD40 agonist directed to the tumor microenvironment through the targeting of FAP-expressing stromal cells. We were able to identify a relevant treatment regimen and showed that the CD8 T cell-DC1 axis drives the main cellular mechanism of action upon IL12 induction at the tumor. Together, the results provide a strong biological rationale for the translation of this therapeutic approach to the clinic. Moreover, the remarkable effects, mediated by human CD40 signaling, upon the combination of radiotherapy with a bispecific surrogate FAP-huCD40 antibody, supports the clinical applicability of this approach to treatment of not only patients with head and neck cancer, but also other cancer types characterized by stromal accumulation of FAP-expressing fibroblasts..

Furthermore, the impact of the therapy on the tumor immune cell landscape and the mechanism of action were explored. The orthotopic model of head and neck cancer (mEERL95) was chosen for several reasons: first, it mimics the human disease regarding the high prevalence of primary tumor recurrences and second, a high level of FAP is found in the mEERL95 stroma. This last characteristic was key to validate the therapeutic efficiency of the FAP-CD40 antibody.

FAP-CD40 only induces CD40 co-stimulation upon when crosslinking to FAP⁺ cells. Such an intrinsic characteristic of this bispecific antibody, to target the CD40 therapeutic antibody to tumor areas where FAP- and CD40-expressing cells co-localize, is the key to avoiding the classical pattern of systemic toxicity described with the use of CD40 agonists. FAP (fibroblast activation protein) is an endopeptidase mainly expressed by protumoral activated fibroblasts from the tumor stroma, but it can be present in other stromal compartments, such as FRC from lymph nodes. By treating the TC-1 tumor model in which minimal efficacy of the combination therapy is consistent with low intratumoral FAP expression, it was found that FAP-mediated crosslinking at the tumor stroma was essential for the anti-tumor effects of the FAP-CD40 antibody. Although, the FAP-CD40 antibody was detected in the tumor area by in vivo fluorescence imaging, we could not formally exclude its presence in the neighboring lymph nodes. However, the equivalent presence of FAP positive cells in the regional lymph nodes of TC-1 and mEERL95 tumors along with their opposite response to the therapy provide indirect evidence that FAP-CD40⁺RT -mediated anti-tumor immune activation occurs in the tumor bed in these studied tumor models. It will be interesting to see how FAP-CD40 performs in immunologically ‘cold’ tumors.

The magnitude of the efficacy reported in mEERL95 tumors and the absence of relapses among responder mice deserve further investigations at the biological level. The long-term survival of the animals treated with the combination of FAP-CD40 and RT and the rejection of the homologous tumor in re-challenged cured mice supported an impact of the combination on long-term immunological memory, which is consistent with the induction of a T cell memory phenotype found in the local lymph nodes. While identification of specific antigens was beyond the scope of the study we ruled out the implication of E7, an obvious antigen given the fact that mEERL95 are HPV⁺. These data were consistent with other reports in which the vaccination with the peptide, alone or in combination with other immunotherapies, augments E7-specific response in tumor preclinical models.

The abrogation of the effects of the combination in T cell-depleted mice, confirmed that such population was crucial for the therapeutic outcome. While CD8 T cells were required for the anti-tumor effect of the combo, CD4 T lymphocytes are essential in the generation of durable responses and prevention of relapses followed by a robust immunological CD8-T memory, as it has been demonstrated in vaccine and viral infection settings (Ahrends et al., Nat. Commun. 2019, 10(1), 5531). Such findings are also supported by previous preclinical studies in which CD4 T cell-mediated immune responses were crucial in anti-CD40 therapy.

The therapeutic effect of the radiotherapy and anti-FAP-CD40 combination therapy was accompanied by a profound remodeling of the tumor immune landscape. The decrease of regulatory T cells and macrophages indicated that the combination prompted a less immunosuppressive context favoring the function of effector T lymphocytes. In this regard, we found an increase in the IFNγ intratumoral levels, that was consistent with a rise in the production of such as cytokine, and low expression levels of PD-1 on restimulated TILs from tumors treated with the combination of FAP-CD40 and radiotherapy. Indeed, IFNγ, which has been postulated as key factor in the CD40-mediated antitumor responses in combination with IL-12 (Garris, Immunity 2018, 49(6), 1149-1161), was induced upon the combination in a FAP-CD40 specific manner. Interestingly, IL-12 neutralization during the effector phase of the response abrogated the therapeutic effect of combination treatment indicating that FAP-CD40 relies on such as cytokine for efficacy. Activated cross-priming dendritic cells are known to be the main producers of IL-12 in humans and mice (Maier et al. Nature 2020, 580, 257-262). In this sense, two noteworthy findings reveal that these cells are crucial in the mechanism of action of the combination. Firstly, the enrichment in an activated crosspriming dendritic cell-specific gene signature upon the combination treatment with FAP-CD40 and radiotherapy in the tumor microenvironment, and secondly, the loss of the anti-tumor response in Batf3ko mice, which lack cDC1 cells. These observations are in agreement with previous studies that reported a dependency of anti-CD40 therapies on crosspriming dendritic cells (Garris, Immunity 2018, 49(6), 1149-1161). Despite macrophages have been described to play a role in the CD40-mediated responses (Beatty et al., Science 2011, 331, 1612-1616), the decrease in their numbers and the key role of IL-12 suggested that the radioimmunotherapeutic effect in our setting is mainly mediated by dendritic cells.

The results that were obtained with the combination treatment in terms of safety and anti-tumor response support the translation of this novel therapeutic approach to the clinic, which combines a gold-standard of care, radiotherapy, with a novel and safe molecularly engineered CD40-targeted immunotherapy.

### Example 3

### In Vivo Anti-Tumor Efficacy of FAP-Targeted Anti-CD40 Antigen Binding Molecules in Combination With Radiotherapy as Shown in the SV2 Lung Tumor Model

### 3.1 Material and Methods

The ability of bispecific FAP-targeted anti-CD40 antigen binding molecules to achieve therapeutic efficacy in combination with a hypofractionated radiation therapy regimen was tested in mice bearing the SV2 tumor cell line, a mouse model for non-small cell lung cancer (NSCLC).

Ten-week-old male C57BL/6J mice were purchased from Charles River Labs. All animal procedures were carried out under the license VD3173.1 which was approved by the Veterinary Authority of the Swiss Canton of Vaud. The SV2 cell line was derived from KP tumor (Kras^{LSL.G12D/wt};p53^{frt/frt}) and obtained from Meylan Lab. The SV2 cell line was transduced with OVA as described previously (Martinez-Usatorre A, Romero P, Generation of affinity ranged antigen-expressing tumor cell lines, Methods Enzymol. 2020, 632, 503-519, doi: 10.1016/bs.mie.2019.12.001. Epub 2019 Dec 18. PMID: 32000912) and was cultured in DMEM medium containing GlutaMAX (Thermo Fisher Scientific) and supplemented with 10% FBS (Life Technologies). Cells were cultured in an incubator at 37° C. and 5% CO₂.

### Tumor Model and In Vivo Treatments

Mice were intravenously injected in the tail vein with 1×10⁶ SV2-OVA cells resuspended in PBS (Thermo Fisher Scientific). At day 12, mice were intravenously injected with 1×10⁵ OT1 cells isolated from naïve spleen. Lungs were irradiated with a hypofractionated regimen consisting in two consecutive doses of 6 Gy at day 13 and 14 post-tumor cell engraftment. Doses were delivered with an Xrad-225CX-PXi Instrument using a 20-mm collimator that allows to locally irradiate the lungs. At day 14 and 18, mice were treated with an intraperitoneal injection of anti-FAP-CD40 (P1AD9139) at 18.3 mg/kg in PBS. Tumor size was followed up using a U-CT system (MILabs) twice per week and the image analysis was performed using Imalytics Preclinical.

### 3.2 Results

### FAP-CD40 in Combination With RT Significantly Delays Tumor Growth in the SV2 Lung Tumor Model

To test the therapeutic potential of the FAP-targeted CD40 bispecific antibody (FAP-CD40), mice were engrafted with 1.5 million SV2-OVA and received 2 suboptimal doses of 6 Gy and 2 doses of FAP-CD40. Before the treatment, mice received 100 000 OT1 i.v.. The treatment schedule is shown in FIG. 3A. It was observed that the combination of RT and anti-FAP-CD40 delays tumor growth compared to the treatment with RT alone (FIG. 3B). The combination also extends survival by 20 more days compared to the untreated mice (FIG. 3C).

What is claimed is:

1. A method of treating an individual having a solid tumor, comprising administering to the individual a therapeutically effective amount of a bispecific agonistic CD40-antigen binding molecule, wherein the bispecific agonistic CD40-antigen binding molecule is administered in combination with radiotherapy, and wherein the bispecific agonistic CD40-antigen binding molecule comprises at least one antigen binding domain capable of specific binding to a tumor-associated antigen.

2. The method of claim 1, wherein the bispecific agonistic CD40-antigen binding molecule comprises at least one antigen binding domain capable of specific binding to CD40 and at least one antigen binding domain capable of specific binding to Fibroblast Activation Protein (FAP).

3. The method of claim 1, wherein the bispecific agonistic CD40-antigen binding molecule is for simultaneous or sequential administration with the radiotherapy.

4. The method of claim 1, wherein the bispecific agonistic CD40-antigen binding molecule is for administration after the radiotherapy.

5. The method of claim 1, wherein the radiotherapy comprises local radiotherapy selected from external beam radiation therapy or brachytherapy.

6. The method of claim 1, wherein the radiotherapy comprises local hypofractionated radiation.

7. The method of claim 6, wherein the radiotherapy comprises local hypofractionated radiation at a dose in the range of 1.8 to 20 Gy.

8. The method of claim 1, wherein the radiotherapy comprises local hypofractionated radiation at a dose of 2 × 6 Gy.

9. The method of claim 1, wherein the solid tumor is selected from the group consisting of head and neck cancer, melanoma, lung cancer, kidney cancer, breast cancer, colon cancer, ovarian cancer, cervical cancer, pancreatic cancer, liver cancer, prostate cancer, bladder cancer, gastric cancer, glioblastoma and sarcomas.

10. The method of claim 9, wherein the solid tumor is head and neck cancer.

11. The method of claim 9, wherein the solid tumor is lung cancer .

12. The method of claim 1, wherein the bispecific agonistic CD40-antigen binding molecule comprises an IgG Fc domain, and wherein the Fc domain comprises one or more amino acid substitution that reduces the binding affinity of the antibody to an Fc receptor, and/or reduces effector function.

13. The of claim 12, wherein the bispecific agonistic CD40-antigen binding molecule comprises an Fc domain of human IgG1 subclass with the amino acid mutations L234A, L235A and P329G, as numbered according to the Kabat EU index.

14. The method of claim 1, wherein the bispecific agonistic CD40-antigen binding molecule comprises at least one antigen binding domain capable of specific binding to CD40 comprising a heavy chain variable region (V_{H}CD40) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}CD40) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

15. The method of claim 1, wherein the bispecific agonistic CD40-antigen binding molecule comprises at least one antigen binding domain capable of specific binding to CD40 comprising a VH comprising the amino acid sequence of SEQ ID NO:7 and a VL comprising the amino acid sequence of SEQ ID NO:8.

16. The method of claim 1, wherein the bispecific agonistic CD40-antigen binding molecule comprises at least one antigen binding domain capable of specific binding to FAP comprising
(a) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or
(b) a heavy chain variable region (V_{H}FAP) comprising (i) CDR-H1 comprising the amino acid sequence of SEQ ID NO:17, (ii) CDR-H2 comprising the amino acid sequence of SEQ ID NO:18, and (iii) CDR-H3 comprising the amino acid sequence of SEQ ID NO:19, and a light chain variable region (V_{L}FAP) comprising (iv) CDR-L1 comprising the amino acid sequence of SEQ ID NO:20, (v) CDR-L2 comprising the amino acid sequence of SEQ ID NO:21, and (vi) CDR-L3 comprising the amino acid sequence of SEQ ID NO:22, or
(c) a heavy chain variable region (V_{H}FAP) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}FAP) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

17. The method of claim 1, wherein the bispecific agonistic CD40-antigen binding molecule comprises at least one antigen binding domain capable of specific binding to FAP comprising a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14.

18. The method of claim 1, wherein the bispecific agonistic CD40-antigen binding molecule comprises at least one antigen binding domain capable of specific binding to FAP comprising
(a) a heavy chain variable region (V_{H}FAP) comprising the amino acid sequence of SEQ ID NO:15 and a light chain variable region (V_{L}FAP) comprising the amino acid sequence of SEQ ID NO:16,
(b) a heavy chain variable region (V_{H}FAP) comprising the amino acid sequence of SEQ ID NO:23 and a light chain variable region (V_{L}FAP) comprising the amino acid sequence of SEQ ID NO:24, or
(c) a heavy chain variable region (V_{H}FAP) comprising the amino acid sequence of SEQ ID NO:31 and a light chain variable region (V_{L}FAP) comprising the amino acid sequence of SEQ ID NO:32.

19. The method of claim 1, wherein the bispecific agonistic CD40-antigen binding molecule comprises
(i) at least one antigen binding domain capable of specific binding to CD40, comprising a heavy chain variable region (V_{H}CD40) comprising the amino acid sequence of SEQ ID NO:7 and a light chain variable region (V_{L}CD40) comprising the amino acid sequence of SEQ ID NO:8, and
(ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:16.

20. The method of claim 1, wherein the bispecific agonistic CD40-antigen binding molecule comprises:
(a) at least two Fab fragments capable of specific binding to CD40 fused at its C-terminus to the N-terminus of a Fc region, and
(b) one antigen binding domain capable of specific binding to FAP fused at its N-terminus to the C-terminus of the Fc region.

21. The method of claim 1, wherein the bispecific agonistic CD40-antigen binding molecule comprises:
a) at least two Fab fragments capable of specific binding to CD40 fused at its C-terminus to the N-terminus of a Fc region, and
(b) a cross-fab fragment capable of specific binding to FAP fused to the C-terminus of the Fc region.

22. The method of claim 21, wherein the bispecific agonistic CD40-antigen binding molecule comprises a cross-fab fragment capable of specific binding to FAP, wherein the VH-Ckappa chain of the cross-fab fragment is fused to the C-terminus of the Fc region.

23. The method of claim 1, wherein the bispecific agonistic CD40-antigen binding molecule comprises four Fab fragments capable of specific binding to CD40, wherein each two Fab fragments are fused to each other and fused at its C-terminus to the N-terminus of a Fc region.

24. The method of claim 1, wherein
(i) the individual has increased survival when treated with the therapeutically effective amount of the bispecific agonistic CD40-antigen binding molecule in combination with radiotherapy compared with an individual who received the bispecific agonistic CD40-antigen binding molecule as monotherapy or who received radiotherapy as monotherapy, or
(ii) wherein the size of the solid tumor in the individual is reduced by more than the additive amount by which the size is reduced by treatment with the bispecific agonistic CD40-antigen binding molecule used as monotherapy and the radiotherapy used as monotherapy.

25. (canceled)

26. (canceled)

27. (canceled)
